(19) United States
(12) Patent Application Publication
(10) Pub. No.: US 2025/0134913 A1
(43) Pub. Date: May 1, 2025

(54) ORAL DOSAGE FORMS OF TRβ AGONIST VK2809 FOR THE TREATMENT OF LIVER DISORDERS AND METHODS OF PREPARING THE SAME

(71) Applicant: Viking Therapeutics, Inc., San Diego, CA (US)

(72) Inventors: Brian Lian, Rancho Santa Fe, CA (US); Geoffrey E. Barker, Carlsbad, CA (US); Maureen Barnes, San Diego, CA (US); William Hoye, San Diego, CA (US)

(21) Appl. No.: 18/836,719

(22) PCT Filed: Feb. 13, 2023
(86) PCT No.: PCT/US2023/012935

§ 371 Date: Aug. 7, 2024

Related U.S. Application Data
(60) Provisional application No. 63/312,650, filed on Feb. 22, 2022.
(60) Provisional application No. 63/311,905, filed on Feb. 18, 2022.

(51) Int. Cl. A61K 31/665; A61K 9/20

(52) U.S. Cl. CPC A61K 31/665; A61K 9/2009; A61K 9/2013; A61K 9/2018; A61K 9/2027; A61K 9/2054; A61K 9/2095

(57) ABSTRACT

The present disclosure is directed to oral dosage forms of TRβ agonist VK2809 and methods of preparing the same. The disclosure provides oral dosage forms of VK2809 with improved stability and solubility properties that allow for storage under normal conditions through the use of spray-drying with one or more polymers selected from the group consisting of: polyvinyl pyrrolidone (PVP), polyvinyl pyrrolidinone-vinyl acetate copolymer (PVP-VA), polyvinyl alcohol (PVA), polyacrylic acid (PAA), polyethylene oxide) (PEO), hydroxypropyl cellulose (HPC), hydroxypropyl methyl cellulose (HPMC), copovidone, poloxamer 407, hypromellose acetate succinate (HPMCAS), polyacrylates and combinations thereof.

### FIELD

The compositions and methods of the present disclosure relate generally to the field of oral dosage forms of thyroid hormone receptor-β (TRβ) agonists for the treatment of liver disorders, fibrotic disease and inflammation.

### BACKGROUND

Thyroid hormones (TH) are synthesized in the thyroid in response to thyroid stimulating hormone (TSH), which is secreted by the pituitary gland in response to various stimulants (e.g., thyrotropin-releasing hormone (TRH) from the hypothalamus). Thyroid hormones are iodinated O-aryl tyrosine analogues excreted into the circulation primarily as 3,3′,5,5′-tetraiodothyronine (T4). T4 is rapidly deiodinated in local tissues by thyroxine 5′-deiodinase to 3,3′,5′-triiodothyronine (T3), which is the most potent TH. T3 is metabolized to inactive metabolites via a variety of pathways, including pathways involving deiodination, glucuronidation, sulfation, deamination, and decarboxylation. Most of the circulating T4 and T3 is eliminated through the liver.

The biological activity of THs is mediated largely through thyroid hormone receptors (TRs). TRs belong to the nuclear receptor superfamily, which, along with its common partner, the retinoid X receptor, form heterodimers that act as ligand-inducible transcription factors. Like other nuclear receptors, TRs have a ligand binding domain and a DNA binding domain and regulate gene expression through ligand-dependent interactions with DNA response elements (thyroid response elements, TREs). Currently, the literature shows that TRs are encoded by two distinct genes (TRα and TRβ), which produce several isoforms through alternative splicing (Williams, Mol. Cell. Biol. 20(22):8329-42 (2000); Nagaya et al., Biochem. Biophys. Res. Commun. 226(2):426-30 (1996)). The major isoforms that have so far been identified are TRα-1, TRα-2, TRβ-1 and TRβ-2. TRα-1 is ubiquitously expressed in the rat with highest expression in skeletal muscle and brown fat. TRβ-1 is also ubiquitously expressed with highest expression in the liver, brain and kidney. TRβ-2 is expressed in the anterior pituitary gland and specific regions of the hypothalamus as well as the developing brain and inner ear. In the rat and mouse liver, TRβ-1 is the predominant isoform (80%). The TR isoforms found in human and rat are highly homologous with respect to their amino acid sequences which suggest that each serves a specialized function.

TH's affect the growth, metabolism and the physiological function of nearly all organs. TH's lower serum cholesterol and triglycerides. However, side effects of TH action include cardiac arrhythmia, bone loss, nervousness, and anxiety.

TRβ agonists may be useful as therapeutics for conditions such as hepatitis, non-alcoholic fatty liver disease, non-alcoholic steatohepatitis (NASH), and a variety of fibrotic disease and disorder. However, some TRβ agonists may be poorly soluble and/or have an undesirable stability profiles. Low aqueous solubility and poor stability of such compounds may require special storage conditions of compounds and may present challenges in preparing formulations that provide adequate exposure to a subject and that do not degrade when stored under normal conditions.

Accordingly, a need exists to provide drug formulations that provide suitable exposure of a drug with poor aqueous solubility to a subject. Furthermore, a need exists for drug formulations with improved stability such that the drug does not degrade under ambient conditions and room temperature storage can be utilized.

### SUMMARY

In a first aspect of the present disclosure, provided herein is an oral dosage form comprising: (A) a composition comprising Compound 1 having the structure:

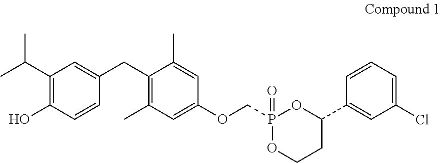

or a pharmaceutically acceptable salt thereof; and one or more polymers selected from the group consisting of: polyvinyl pyrrolidone (PVP), polyvinyl pyrrolidinone-vinyl acetate copolymer (PVP-VA), polyvinyl alcohol (PVA), polyacrylic acid (PAA), poly(ethylene oxide) (PEO), hydroxypropyl cellulose (HPC), hydroxypropyl methyl cellulose (HPMC), copovidone, poloxamer 407, hypromellose acetate succinate (HPMCAS), polyacrylates and combinations thereof, and (B) one or more ductile fillers, brittle fillers, disintegrants, glidants, lubricants, or combinations thereof. In some embodiments, the polymer may be polyvinyl pyrrolidinone-vinyl acetate copolymer (PVP-VA).

In some embodiments of the first aspect, Compound 1 and the one or more polymers may be combined to form a spray dried dispersion. In other embodiments, Compound 1 and the one or more polymers may be combined to form a hot melt extrusion.

In some embodiments of the first aspect, the mass ratio of Compound 1 and the one or more polymers in the composition may be from 1:10 to 10:1. In other embodiments, the mass ratio of Compound 1 and the one or more polymers in the composition may be from 1:1 to 1:4. In some specific embodiments, the mass ratio of Compound 1 and the one or more polymers in the composition may be 1:3.

In some embodiments of the first aspect, the composition may comprise 5% to 25% by weight of the dosage form. In other embodiments, the composition may comprise 10% to 15% by weight of the dosage form.

In some embodiments of the first aspect, the ductile filler is selected from microcrystalline cellulose and silicified microcrystalline cellulose. In some specific embodiments, the ductile filler is microcrystalline cellulose.

In some embodiments of the first aspect, the microcrystalline cellulose may comprise 40% to 60% by weight of the dosage form. In other embodiments, the microcrystalline cellulose may comprise 45% to 55% by weight of the dosage form. In some embodiments, the microcrystalline cellulose may comprise 50% by weight of the dosage form.

In some embodiments of the first aspect, the brittle filler may comprise lactose monohydrate, spray dried lactose, anhydrous lactose, lactose monohydrate, anhydrous lactose, mannitol, and combinations thereof. In some embodiments, the brittle filler may comprise lactose monohydrate, anhydrous lactose, or mannitol.

In some embodiments of the first aspect, the brittle filler may comprise 10% to 40% by weight of the dosage form. In other embodiments, the brittle filler may comprise 20% to 30% by weight of the dosage form. In some embodiments, the brittle filler may comprise 25% by weight of the dosage form.

In some embodiments of the first aspect, the disintegrant may be selected from carmellose calcium, carboxymethylstarch sodium, croscarmellose sodium, starch, pregelatinized starch, sodium starch glycolate, crosslinked polyvinylpyrrolidone crospovidone, hydroxypropylcellulose, magnesium aluminometasilicate, and polacrilin potassium. In some embodiments, the disintegrant may be croscarmellose sodium or crospovidone.

In some embodiments of the first aspect, the disintegrant may comprise 5% to 15% by weight of the dosage form. In other embodiments, the disintegrant may comprise 10% by weight of the dosage form.

In some embodiments of the first aspect, the lubricant may be selected from the group consisting of stearic acid, talc, glyceryl behenate, sodium stearyl fumarate and magnesium stearate. In some specific embodiments, the lubricant may be magnesium stearate.

In some embodiments of the first aspect, the lubricant may comprise 0.1% to 3% by weight of the dosage form. In other embodiments, the lubricant may comprise 0.1% to 1% by weight of the dosage form. In some embodiments, the lubricant may comprise 5% by weight of the dosage form.

In some embodiments of the first aspect, the glidant may silicon dioxide, starch, or talc. In some specific embodiments, the glidant may be silicon dioxide.

In some embodiments of the first aspect, wherein the glidant comprises 0.1% to 3% by weight of the dosage form. In other embodiments, the glidant may comprise 0.1% to 1% by weight of the dosage form. In some embodiments, the glidant may comprise 5% by weight of the dosage form.

In some embodiments of the first aspect, the oral dosage for described herein may comprise: (a) an intragranular portion comprising. (i) Compound 1, or a pharmaceutically acceptable salt thereof, and the one or more polymers; (ii) a ductile filler; (iii) a brittle filler; (iv) a disintegrant; (v) a glidant; and (vi) a lubricant; and (b) an extragranular portion comprising a (i) disintegrant; and (ii) a lubricant.

In some embodiments of the first aspect, the dosage form may be characterized by having from 90% to 100% of the original amount of Compound 1 after 1 month of storage at 50° C. and ambient relative humidity (RH). In other embodiments, the dosage form is characterized by having from 95% to 100% of the original amount of Compound 1 after 1 month of storage at 50° C. and ambient relative humidity (RH). In yet other embodiments, the dosage form may be characterized by having from 90% to 100% of the original amount of Compound 1 after 2 months of storage at 50° C. and ambient relative humidity (RH). In other embodiments, the dosage form may be characterized by having from 95% to 100% of the original amount of Compound 1 after 2 months of storage at 50° C. and ambient relative humidity (RH).

In a second aspect of the present disclosure, provided herein is a method of preparing an oral dosage described herein comprising the steps of: (a) preparing a pregranulation pre-blend comprising (a) a composition comprising Compound 1 and the one or more polymers; (ii) a ductile filler; (iii) a brittle filler; (iv) a disintegrant; and (v) a glidant; (b) blending the pre-blend composition; (c) further adding lubricant to the pregranulation pre-blend; (d) slugging the pregranulation pre-blend; and (e) granulating the pregranulation pre-blend to form granulated material.

In some embodiments, the method may further comprise the steps of (f) adding a disintegrant to the granulated material; (g) further blending the granulated material; (h) further adding lubricant to the granulated material and blend to form a final blend; and (i) compressing the final blend into a dosage form.

In a third aspect of the present disclosure, provided herein is a method of preventing, treating, or ameliorating one or more fatty liver diseases in a subject in need thereof comprising administering to said subject in need thereof, the method comprising administering the oral dosage forms described herein to the subject.

In some embodiments, the fatty liver disease may be selected from the group consisting of steatosis, non-alcoholic fatty liver disease, and non-alcoholic steatohepatitis.

In some embodiments, the method may comprise administration of a second pharmaceutical agent. In some embodiments, the second pharmaceutical agent may be administered sequentially or simultaneously.

In some embodiments, the method may result in the prevention, treatment, or amelioration, of a fibrosis, fibrotic condition, or fibrotic symptom. In some embodiments, the method may result in the reduction in the amount of extracellular matrix proteins present in one or more tissues of said subject. In some embodiments, the method may result in the reduction in the amount of collagen present in one or more tissues of said subject. In some embodiments, the method may result in the reduction in the amount of Type I, Type Ia, or Type III collagen present in one or more tissues of said subject.

### BRIEF DESCRIPTION OF THE DRAWINGS

FIG. 1 shows the relative scattering absorbance with measured concentration of Compound 1 from a stock solution into biorelevant media at 37° C.

FIG. 2 shows the dissolution of Compound 1 Spray Dried Dispersions in PBS buffer with 0.5% simulated intestinal fluid at pH 6.5 after exposure to 0.01 N HCl for 30 minutes as measured with UV-Vis probes.

### DETAILED DESCRIPTION

The present disclosure provides oral dosage formulations of Compound 1:

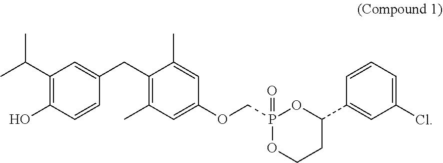

Compound 1 is a low solubility, lipophilic prodrug compound in development for the treatment of chronic liver diseases including non-alcoholic fatty liver disease (NAFLD). Compound 1 may be prepared according to known methods, including those described in U.S. Pat. No. 7,829,552, which is incorporated herein by reference in its entirety. The current clinical dosage form is an encapsulated PEG-based formulation that provides adequate exposure but requires cold chain storage due to chemical stability challenges. Provided herein are immediate release tablet formulations that comprise solid amorphous spray dried dispersions (SDD) of Compound 1 that matches or exceeds the exposure provided by the current formulation, with improved stability such that room temperature storage can be utilized.

### Definitions

The term “mammal” is used in its usual biological sense. Thus, it specifically includes humans and non-human mammals such as dogs, cats, horses, donkeys, mules, cows, domestic buffaloes, camels, llamas, alpacas, bison, yaks, goats, sheep, pigs, elk, deer, domestic antelopes, and non-human primates as well as many other species.

“Subject” as used herein, means a human or a non-human mammal including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

“Subject suspected of having” means a subject exhibiting one or more clinical indicators of a disease or condition. In certain embodiments, the disease or condition is one or more fibroses, fibrotic conditions, or fibrotic symptoms. In certain embodiments, the disease or condition is scleroderma. In certain embodiments, the disease or condition is non-alcoholic steatohepatitis (NASH). In certain embodiments, the disease or condition is cirrhosis. In certain embodiments, the disease or condition is non-alcoholic fatty liver disease (NAFLD). In certain embodiments, the disease or condition is idiopathic pulmonary fibrosis. In certain embodiments, the disease or condition is atherosclerosis. In certain embodiments, the disease or condition is hepatitis, alcoholic fatty liver disease, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, scleroderma, pulmonary fibrosis, diffuse parenchymal lung disease, idiopathic interstitial fibrosis, diffuse interstitial fibrosis; interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-polymyositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis, or symptoms or sequelae thereof, or other diseases or conditions resulting in the excessive deposition of extracellular matrix components.

As used herein, “fibrosis” refers to the abnormal deposition of extracellular matrix proteins. Such proteins include but are not limited to collagen, elastin, fibronectin, laminin, keratin, keratin, keratin sulfate, fibrin, perlecan, agrin, or agrecan. As used herein, “collagen” refers to any one of the subtypes of collagen, including but not limited to Type I, II, III, IV, V, VI, VII, VIII, IX, X, XI, XII, XIII, XIV, XV, XVI, XVII, or XVIII. Exemplary collagen types and subtypes especially include Type I, Type Ia, Type II, Type III, Type IV, and Type V. As used herein, fibrosis may occur by itself or as a symptom or sequela of another condition. As used herein, fibrosis may result from a genetic condition, a genetic predisposition, an environmental insult, an injury, healing of an injury, an autoimmune condition, or a chronic inflammation, a chronic inflammatory condition, or another condition leading to abnormal or excessive deposition of extracellular matrix components. Fibrosis as referred to herein may be assessed by assaying for, or determining the presence or level of, one or more biomarkers. Biomarkers for the presence of fibrosis include, but are not limited to, expression of the Col1a1, Col3a1, ACTA2, ENPP2, and/or LGALS1 genes or any combination or product thereof. Diagnosis or assessment of fibrosis may further be made by determination of the presence or level of type I collagen and/or hydroxyproline or any combination or product thereof. Diagnosis or assessment of fibrosis may also be made by histological, histochemical, or immunohistochemical analysis of one or more samples from a subject.

“Glycogen storage disease” means any one or more of a group of disorders marked by dysfunction in the synthesis, transport, or utilization of glycogen, generally due to the loss of a necessary enzyme activity. Glycogen storage diseases are generally classified by type according to their symptoms and etiologies. Known types include GSD type 0 (aglycogenesis, glycogen synthase deficiency); GSD type 1 (von Gierke disease, glucose-6-phosphatase translocase/transporter deficiency, GSD I); GSD type 2 (Pompe disease, alpha-1-4-glucosidase deficiency, GSD II); GSD type 3 (Cori disease, Forbes disease, limit dextrinosis, debranching enzyme disease; amylo-1-6-glucosidase deficiency due to loss of glucosidase, and/or transferase activity, GSD III); GSD type 4 (Andersen disease, glycogen phosphorylase deficiency, brancher deficiency, amylopectinosis, glycogen branching enzyme deficiency; amylo-1,4 to 1,6 transglucosidase deficiency, GSD IV); GSD type 5 (McArdle disease; glycogen phosphorylase (muscle type) deficiency, GSD V); GSD type 6 (Hers disease; glycogen phosphorylase E (liver type) deficiency, GSD VI); GSD type 7 (Tarui disease; phosphofructokinase deficiency, GSD VII); GSD type 8, 9 (GSD with phosphorylase activation system defects; phosphorylase kinase (liver or muscle isoforms) deficiency, GSD VIII and GSD IX); GSD type 10 (cyclic AMP-dependent kinase deficiency, GSD X); GSD type 11 (Fanconi-Bickel syndrome; glucose transporter type 2 (GLUT2) deficiency, GSD XI); and GSD type 12 (aldolase A deficiency, GSD XII). Subtypes of glycogen storage diseases are also known, in particular GSD 1a, which results from mutations in the gene for glucose-6-phosphatase (G6PC) and leads to, among other symptoms, the excess accumulation of glycogen and lipids in liver tissue, hepatomegaly, hepatic adenomas, and hepatocellular carcinoma. Symptoms of glycogen storage diseases may include elevated or reduced blood sugar, insulin insensitivity, myopathies, as well as hepatic symptoms such as steatosis, hyperlipidemia, hypercholesterolemia, cardiomegaly, hepatomegaly, fibrosis, cirrhosis, hepatocellular adenoma, and hepatocellular carcinoma. Symptoms may also include insulin insensitivity, elevated or reduced blood glucose, renal dysfunction, and/or fibrosis.

As used herein, “inflammatory disease” refers to a disease or disorder that is characterized by inflammation. Exemplary inflammatory diseases include, but are not limited to, acne, acid reflux/heartburn, age related macular degeneration (AMD), allergy, allergic rhinitis, Alzheimer's disease, amyotrophic lateral sclerosis, anemia, appendicitis, arteritis, arthritis, asthma. atherosclerosis, autoimmune disorders, balanitis, blepharitis, bronchiolitis, bronchitis, a bullous pemphigoid, burn, bursitis, cancer, cardiac arrest, carditis, celiac disease, cellulitis, cervicitis, cholangitis, cholecystitis, chorioamnionitis, chronic obstructive pulmonary disease (COPD), cirrhosis, colitis, congestive heart failure, conjunctivitis, cyclophosphamide-induced cystitis, cystic fibrosis, cystitis, common cold, dacryoadenitis, dementia, dermatitis, dermatomyositis, diabetes, diabetic neuropathy, diabetic retinopathy, diabetic nephropathy, diabetic ulcer, digestive system disease, eczema, emphysema, encephalitis, endocarditis, endometritis, enteritis, enterocolitis, epicondylitis, epididymitis, fasciitis, fibromyalgia, fibrosis, fibrositis, gastritis, gastroenteritis, gingivitis, glomerulonephritis, glossitis, heart disease, heart valve dysfunction, hepatitis, hidradenitis suppurativa, Huntington's disease, hyperlipidemic pancreatitis, hypertension, ileitis, infection, inflammatory bowel disease, inflammatory cardiomegaly, inflammatory neuropathy, insulin resistance, interstitial cystitis, interstitial nephritis, iritis, ischemia, ischemic heart disease, keratitis, keratoconjunctivitis, laryngitis, lupus nephritis, mastitis, mastoiditis, meningitis, metabolic syndrome (syndrome X), a migraine, multiple sclerosis, myelitis, myocarditis, myositis, nephritis, non-alcoholic steatohepatitis, obesity, omphalitis, oophoritis, orchitis, osteochondritis, osteopenia, osteomyelitis, osteoporosis, osteitis, otitis, pancreatitis, Parkinson's disease, parotitis, pelvic inflammatory disease, pemphigus vularis, pericarditis, peritonitis, pharyngitis, phlebitis, pleuritis, pneumonitis, polycystic nephritis, proctitis, prostatitis, psoriasis, pulpitis, pyelonephritis, pylephlebitis, renal failure, reperfusion injury, retinitis, rheumatic fever, rhinitis, salpingitis, sarcoidosis, sialadenitis, sinusitis, spastic colon, stenosis, stomatitis, stroke, surgical complication, synovitis, tendonitis, tendinosis, tenosynovitis, thrombophlebitis, tonsillitis, trauma, traumatic brain injury, transplant rejection, trigonitis, tuberculosis, tumor, urethritis, ursitis, uveitis, vaginitis, vasculitis, and vulvitis. Inflammation as referred to herein may be assessed by assaying for, or determining the presence or level of, one or more biomarkers. Biomarkers for the presence of inflammation include, but are not limited to, expression of the TNF, CARD15, IL4R, IL23R, CTLA4, ANXA1, ANXA2, LGALS3, and/or PTPN22 genes or any combination or product thereof.

The term “atherosclerosis” refers to a condition characterized by irregularly distributed lipid deposits in the intima of large and medium-sized arteries wherein such deposits provoke fibrosis and calcification. Atherosclerosis raises the risk of angina, stroke, heart attack, or other cardiac or cardiovascular conditions.

“Subject in need thereof” means a subject identified as in need of a therapy or treatment.

A therapeutic effect relieves, to some extent, one or more of the symptoms of a disease or disorder, and includes curing the disease or disorder. “Curing” means that the symptoms of active disease are eliminated. However, certain long-term or permanent effects of the disease may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period of time, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Modulation” means a perturbation of function or activity. In certain embodiments, modulation means an increase in gene expression. In certain embodiments, modulation means a decrease in gene expression. In certain embodiments, modulation means an increase or decrease in total serum levels of a specific protein. In certain embodiments, modulation means an increase or decrease in free serum levels of a specific protein. In certain embodiments, modulation means an increase or decrease in total serum levels of a specific non-protein factor. In certain embodiments, modulation means an increase or decrease in free serum levels of a specific non-protein factor. In certain embodiments, modulation means an increase or decrease in total bioavailability of a specific protein. In certain embodiments, modulation means an increase or decrease in total bioavailability of a specific non-protein factor.

“Administering” means providing a pharmaceutical agent or composition to a subject, and includes, but is not limited to, administering by a medical professional and self-administering.

The term “agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances.

“Pharmaceutical agent” means a substance that provides a therapeutic effect when administered to a subject.

“Pharmaceutical composition” means a mixture of substances suitable for administering to an individual that includes a pharmaceutical agent. For example, a pharmaceutical composition may comprise a modified oligonucleotide and a sterile aqueous solution.

The term “pharmaceutically acceptable salt” refers to salts that retain the biological effectiveness and properties of the compounds with which they are associated and, which are not biologically or otherwise undesirable. In many cases, the compounds herein are capable of forming acid and/or base salts by virtue of the presence of phenol and/or phosphonate groups or groups similar thereto. One of ordinary skill in the art will be aware that the protonation state of any or all of these compounds may vary with pH and ionic character of the surrounding solution, and thus the present disclosure contemplates multiple charge states of each compound. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

### Pharmaceutical Compositions

The compounds useful as described above can be formulated into pharmaceutical compositions for use in treatment of the conditions described herein. In some embodiments, pharmaceutical compositions comprising Compound 1 may be formulated for oral administration. In some specific embodiments, pharmaceutical compositions comprising Compound 1 may be formulated as a tablet. Some embodiments of the pharmaceutical compositions described herein comprise: (a) a safe and therapeutically effective amount of Compound 1, or pharmaceutically acceptable salts thereof; and (b) a pharmaceutically acceptable carrier, diluent, excipient or combination thereof.

The compositions described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often that once daily, and may be administered more than once during a course of therapy.

The actual unit dose of Compound 1 described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight. In some embodiments, the dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight. In some embodiments, the actual unit dose is 0.05, 0.07, 0.1, 0.3, 1.0, 3.0, 5.0, 10.0 or 25.0 mg/kg of body weight. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, or from about 20 mg to about 300 mg, or from about 200 mg to about 2000 mg. In some embodiments, the actual unit dose is 0.1 mg. In some embodiments, the actual unit dose is 0.5 mg. In some embodiments, the actual unit dose is 1 mg. In some embodiments, the actual unit dose is 1.5 mg. In some embodiments, the actual unit dose is 2 mg. In some embodiments, the actual unit dose is 2.5 mg. In some embodiments, the actual unit dose is 3 mg. In some embodiments, the actual unit dose is 3.5 mg. In some embodiments, the actual unit dose is 4 mg. In some embodiments, the actual unit dose is 4.5 mg. In some embodiments, the actual unit dose is 5 mg. In some embodiments, the actual unit dose is 10 mg. In some embodiments, the actual unit dose is 20 mg. In some embodiments, the actual unit dose is 25 mg. In some embodiments, the actual unit dose is 250 mg or less. In some embodiments, the actual unit dose is 100 mg or less. In some embodiments, the actual unit dose is 70 mg or less.

In some embodiments, Compound 1 is administered at a dose in the range of about 1-50 mg/m² of the body surface area. In some embodiments, Compound 1 is administered at a dose in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, 2.5-22.5, or 9.5-21.5 mg/m², of the body surface area. In some embodiments, Compound 1 is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, Compound 1 is administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, Compound 1 is administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, the Compound 1 dose is about 0.1 mg-100 mg, 0.1 mg-50 mg, 0.1 mg-20 mg, 0.1 mg-10 mg, 0.5 mg-100 mg, 0.5 mg-50 mg, 0.5 mg-20 mg, 0.5 mg-10 mg, 1 mg-100 mg, 1 mg-50 mg, 1 mg-20 mg, 1 mg-10 mg, 2.5 mg-50 mg, 2.5 mg-20 mg, 2.5 mg-10 mg, or about 2.5 mg-5 mg. In some embodiments, the TR-β agonist compound dose is about 5 mg-300 mg, 5 mg-200 mg, 7.5 mg-200 mg, 10 mg-100 mg, 15 mg-100 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, the amount of Compound 1 administered is about 20 mg-60 mg, 27 mg-60 mg, 20 mg-45 mg, or 27 mg-45 mg. In some embodiments, the amount of Compound 1 administered is about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the Compound 1 dose is greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the Compound 1 dose is about less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the Compound 1 dose is about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 225 mg, about 250 mg, about 275 mg, or about 300 mg.

### Tablet Formulations

Compound 1 may be combined with one or more polymers and then further formulation into a desired dosage form. In some embodiments, Compound 1 is in the form of a spray dried dispersion (SDD). In other embodiments, Compound 1 is in the form of a hot melt extrusion. In some embodiments, the dosage form is an oral dosage form. In some specific embodiments, the oral dosage form is a tablet. In other embodiments, the oral dosage from is a capsule.

In some embodiments, the polymer for preparation of the spray dried dispersion comprising Compound 1 may be selected from one or more of polyvinyl pyrrolidone (PVP) polyvinyl pyrrolidone-vinyl acetate copolymer (PVP-VA), polyvinyl alcohol (PVA), polyacrylic acid (PAA), poly(ethylene oxide) (PEO), hydroxypropyl cellulose (HPC), hydroxypropyl methyl cellulose (HPMC), copovidone, poloxamer 407, hypromellose acetate succinate (HPMCAS), Eudragit®, and polyacrylates. In some embodiments, the polymer may be PVP. In some embodiments, the polymer may be PVP-VA In other embodiments the polymer may be HPMCAS in yet other embodiments, the polymer may be HPMC in some embodiments, the polymer may be Eudragit®. In some embodiments, the polymer for the preparation of the spray dried dispersion of Compound 1 may be a combination of PVP-VA and poloxamer 407.

Spray dried dispersions of Compound 1 with one or more polymers may be prepared by combining Compound 1 and polymer in a suitable solvent and then spraying the feed into a hot drying medium to remove the solvent. Preparing a spray dried dispersion (SDD) of Compound 1 and one or more polymers may increase the aqueous solubility (and consequently the bioavailability) of Compound 1 and may increase the stability of Compound 1 such that storage under normal conditions is possible.

In some embodiments, the mass ratio of Compound 1 to polymer in the SDD is from about 1:10 to about 10:1. For example, in some embodiments, the mass ratio of Compound 1 to polymer in the SDD is about 1:1, 1.5:1, 2:1, 2.5:1, 3:1, 3.5:1, 4:1, 4.5:1 or 5:1. In some embodiments, the mass ratio of Compound 1 to polymer in the SDD can be in the range of about 1:1 to 5:1, 1.5:1 to 5:1, 2:1 to 4:1, 2.5:1 to 3.5:1 or 3:1 to 5:1. In some embodiments, the mass ratio of Compound 1 to polymer in the SDD can be in the range of about 1:1 to 1:5, 1:1 to 1:4, 1:1 to 1:3, or 1:2 to 1:4. In some embodiments, the mass ratio of Compound 1 to polymer in the SDD can be about 1:3.

Compound 1 may be formulated into a tablet for oral administration to a subject in need thereof. In some embodiments, Compound 1 in the tablet may be present at about 5%, 6%, 7%, 8%, 9%, 10%, 11%, 12%, 13%, 14%, 15%, 16%, 17%, 18%, 19%, 20%, 21%, 22%, 23%, 24%, or 25% by weight of the tablet, or within a range defined by any two of the aforementioned amounts. For example, in some embodiments, Compound 1 in the tablet may be present in about 5% to 25%, 10% to 20%, 5% to 15%, or 10% to 15% by weight.

The tablet formulation of Compound 1 described herein may further comprise a ductile filler. In some embodiments, the ductile filler in the tablet may be present at about 30%, 35%, 40%, 45%, 50%, 55%, 60%, 65%, or 70% by weight of the tablet, or within a range defined by any two of the aforementioned amounts. For example, in some embodiments, the ductile filler in the tablet may be present in about 30% to 70%, 40% to 60%, 45% to 55%, or 50% to 55% by weight. In some embodiments, the ductile filler may be microcrystalline cellulose. In other embodiments, the ductile filler may comprise silicified microcrystalline cellulose.

The tablet formulation of Compound 1 described herein may further comprise a brittle filler. In some embodiments, the brittle filler in the tablet may be present at about 10%, 15%, 20%, 25%, 30%, 35%, or 40% by weight of the tablet, or within a range defined by any two of the aforementioned amounts. For example, in some embodiments, the brittle filler in the tablet may be present in about 10% to 40%, 10% to 30%, 15% to 30%, or 20% to 30% by weight. In some embodiments, the brittle filler may be lactose monohydrate, spray dried lactose, anhydrous lactose, lactose monohydrate, anhydrous lactose, mannitol, or combinations thereof. In some specific embodiments, the brittle filler may be lactose monohydrate. In other specific embodiments, the brittle filler may be mannitol. In yet other specific embodiments, the brittle filler may be anhydrous lactose.

The tablet formulations of Compound 1 described herein may further comprise a disintegrant. In some embodiments, the disintegrant in the tablet may be present at about 5%, 6%, 7%, 8%, 9%, 10%, 11%, 12%, 13%, 14%, 15%, 16%, 17%, 18%, 19%, or 20% by weight of the tablet, or within a range defined by any two of the aforementioned amounts. For example, in some embodiments, the disintegrant in the tablet may be present in about 5% to 20%, 5% to 15%, 10% to 15%, or 10% to 20% by weight. In some embodiments, the disintegrant may be carmellose calcium, carboxymethylstarch sodium, croscarmellose sodium, starch, pregelatinized starch, sodium starch glycolate, crosslinked polyvinylpyrrolidone crospovidone, hydroxypropylcellulose, magnesium aluminometasilicate, polacrilin potassium or any combination thereof. In some specific embodiments, the disintegrant may be croscarmellose sodium. In other embodiments, the disintegrant may be crospovidone.

The tablet formulations of Compound 1 described herein may further comprise a glidant. In some embodiments, the glidant in the tablet may be present at about 0.1%, 0.2%, 0.3%, 0.4%, 0.5%, 0.6%, 0.7%, 0.8%, 0.9%, 1.00%, 1.1%, 1.2%, 1.3%, 1.4%, 1.5%, 1.6%, 1.7%, 1.8%, 1.9%, or 2.0% by weight of the tablet, or within a range defined by any two of the aforementioned amounts. For example, in some embodiments, the glidant in the tablet may be present in about 0.1% to 2.0%, 0.3% to 2.0%, 0.1% to 1.0%, or 0.5% to 1.0% by weight. In some embodiments, the glidant may be silicon dioxide, starch, talc or any combination thereof. In some specific embodiments, the glidant may be silicon dioxide.

The tablet formulations of Compound 1 described herein may further comprise a lubricant. In some embodiments, the lubricant in the tablet may be present at about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% by weight of the tablet, or within a range defined by any two of the aforementioned amounts. For example, in some embodiments, the lubricant in the tablet may be present in about 0.1% to 2.0%, 0.3% to 2.0%, 0.1% to 1.0%, or 0.5% to 1.0% by weight. In some embodiments, the lubricant may be stearic acid, talc, glyceryl behenate, sodium stearyl fumarate, magnesium stearate, or any combination thereof. In some specific embodiments, the lubricant may be magnesium stearate.

The tablet formulations of Compound 1 described herein may comprise an intragranular component and an extragranular component. For example, in some embodiments, the tablet formulation may comprise an intragranular component comprising Compound 1 and one or more polymers, a ductile filler; a brittle filler; a disintegrant; a glidant; and a lubricant; an extragranular component comprising a disintegrant and a lubricant. In some embodiments, the disintegrant in the intragranular component may be the same as the disintegrant in the extragranular component. In other embodiments, the disintegrant in the intragranular component may be different the disintegrant in the extragranular component. In some embodiments, the lubricant in the intragranular component may be the same as the lubricant in the extragranular component. In other embodiments, the lubricant in the intragranular component may be different the lubricant in the extragranular component.

In some embodiments, the tablet formulations comprising Compound 1 described herein comprises about 5%, 6%, 7%, 8%, 9%, 10%, 11%, 12%, 13%, 14%, 15%, 16%, 17%, 18%, 19%, 20%, 21%, 22%, 23%, 24%, or 25% of the one or more polymers, or within a range defined by any two of the aforementioned amounts. For example, in some embodiments, the one or more polymers in the tablet may be present in about 5% to 25%, 10% to 20%, 5% to 15%, or 10% to 15% by weight.

Unlike current clinical formulations of Compound 1, the tablet formulations comprising Compound 1 described herein are stable under standard storage conditions. For example, the tablet formulations described herein show minimal degradation as measured by HPLC upon storage in high density polyethylene bottles for one, two, and three months at 40° C. or 50° C. and ambient relative humidity (RH). In some embodiments, the amount of Compound 1 in the tablet formulation may comprise 95%, 96%, 97%, 98%, 99%, or more by weight of the original amount of Compound 1 in the formulation upon storage in a closed bottle for 1 month at 40° C. and ambient RH. In some embodiments, the amount of Compound 1 in the tablet formulation may comprise 95%, 96%, 97%, 98%, 99% or more by weight of the original amount of Compound 1 in the formulation upon storage in a closed bottle for 1 month at 50° C. and ambient RH. In some embodiments, the amount of Compound 1 in the tablet formulation may comprise 95%, 96%, 97%, 98%, 99% or more by weight of the original amount of Compound 1 in the formulation upon storage in a closed bottle for 2 months at 40° C. and ambient RH. In some embodiments, the amount of Compound 1 in the tablet formulation may comprise 95%, 96%, 97%, 98%, 99% or more by weight of the original amount of Compound 1 in the formulation upon storage in a closed bottle for 2 months at 50° C. and ambient RH. In some embodiments, the amount of Compound 1 in the tablet formulation may comprise 95%, 96%, 97%, 98%, 99% or more by weight of the original amount of Compound 1 in the formulation upon storage in a closed bottle for 3 months at 40° C. and ambient RH. In some embodiments, the amount of Compound 1 in the tablet formulation may comprise 95%, 96%, 97%, 98%, 99% or more by weight of the original amount of Compound 1 in the formulation upon storage in a closed bottle for 3 months at 50° C. and ambient RH.

### Second Pharmaceutical Agents

The dosage forms of Compound 1 disclosed herein may be administered in combination with one or more second pharmaceutical agents. In some embodiments, the dosage forms of Compound 1 disclosed herein may be administered in combination with one second pharmaceutical agent. In some embodiments, the compounds described above may be administered in combination with two second pharmaceutical agents. In some embodiments, the compounds described above may be administered in combination with three or more second pharmaceutical agents.

In some embodiments, the dosage forms of Compound 1 presented herein may be administered simultaneously with one or more second pharmaceutical agents. In other embodiments, the dosage forms of Compound 1 of the present disclosure may be administered sequentially with one or more second pharmaceutical agents. In some embodiments, Compound 1 and the second pharmaceutical agent are included together in the dosage forms described herein.

In one aspect, the dosage forms of Compound 1 presented herein may be administered in combination with a peroxisome proliferator-activated receptor (PPAR) modulator. PPAR modulators are pharmaceutical compounds that may be used e.g., to lower triglyceride levels and blood sugar levels in a subject. PPAR modulators may be classified as PPARα modulators, PPARγ modulators, or PPARδ agonists. In some embodiments, the PPAR modulator may be

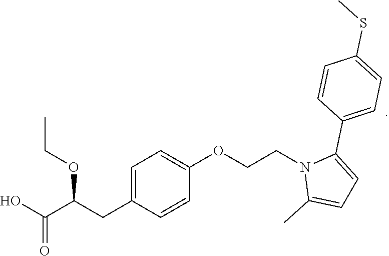 [SMILES: CCO[C@@H](Cc1ccc(OCCn2c(C)ccc2-c2ccc(SC)cc2)cc1)C(=O)O]

In some embodiments, the PPAR modulator may be

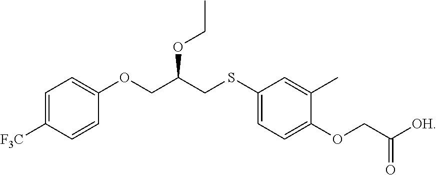 [SMILES: CCO[C@H](COc1ccc(C(F)(F)F)cc1)CSc1ccc(OCC(=O)O)c(C)c1]

In some embodiments, the PPAR modulator may be

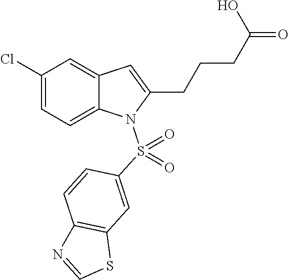 [SMILES: O=C(O)CCCc1cc2cc(Cl)ccc2n1S(=O)(=O)c1ccc2ncsc2c1]

In some embodiments, the PPAR modulator may be

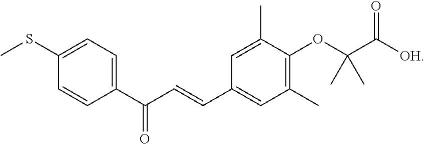 [SMILES: CSc1ccc(C(=O)/C=C/c2cc(C)c(OC(C)(C)C(=O)O)c(C)c2)cc1]

In some embodiments, the PPAR modulator may be a pharmaceutically acceptable salt or prodrug of any of the foregoing.

In some embodiments, the dosage forms of Compound 1 presented herein herein may be administered in combination with a fibric acid derivative. Fibric acid derivatives are a class of lipid-lowering drugs that have the ability to lower a subject's lipid profile. In some embodiments, the fibric acid derivative may be fenofibrate. In some embodiments, the fibric acid derivative may be gemfibrozil. In some embodiments, the fibric acid derivative may be fenofibric acid. In some embodiments, the fibric acid derivative may be clofibrate. In some embodiments, the fibric acid derivative may be a pharmaceutically acceptable salt or prodrug of any of the foregoing.

In some embodiments, the dosage forms of Compound 1 presented herein may be administered in combination with a bile acid receptor modulator. Bile acid receptors include, but are not limited to FXR (farnesoid X receptor) and TGR5. In some embodiments, the bile acid receptor modulator may be

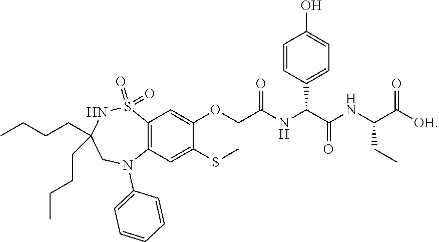 [SMILES: CCCCC1(CCCC)CN(c2ccccc2)c2cc(SC)c(OCC(=O)N[C@@H](C(=O)N[C@@H](CC)C(=O)O)c3ccc(O)cc3)cc2S(=O)(=O)N1]

In some embodiments, the bile acid receptor modulator may be

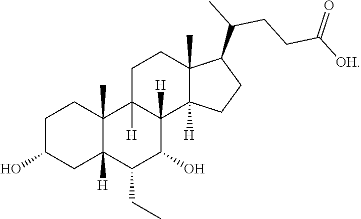

In some embodiments, the bile acid receptor modulator may be

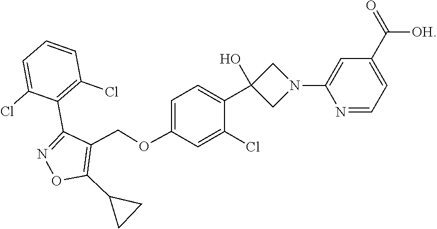 [SMILES: O=C(O)c1ccnc(N2CC(O)(c3ccc(OCc4c(-c5c(Cl)cccc5Cl)noc4C4CC4)cc3Cl)C2)c1]

In some embodiments, the bile acid receptor modulator may be

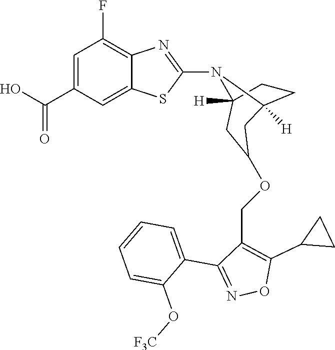 [SMILES: O=C(O)c1cc(F)c2nc(N3[C@@H]4CC[C@H]3CC(OCc3c(-c5ccccc5OC(F)(F)F)noc3C3CC3)C4)sc2c1]

(tropifexor). In some embodiments, the bile acid modulator may be a pharmaceutically acceptable salt or prodrug of any of the foregoing.

In some embodiments, the dosage forms of Compound 1 presented herein may be administered in combination with a bile acid receptor modulator. In some embodiments, the bile acid receptor modulator may be selected from the group consisting of an FXR agonist, an FXR antagonist, a TGR agonist, and a dual FXR/TGR agonist. In some embodiments, the bile receptor acid modulator may be selected from a compound disclosed in Xu, J. Med. Chem. 2016, 59, 6553-6579, which is incorporated herein by reference in its entirety, including compounds selected from:

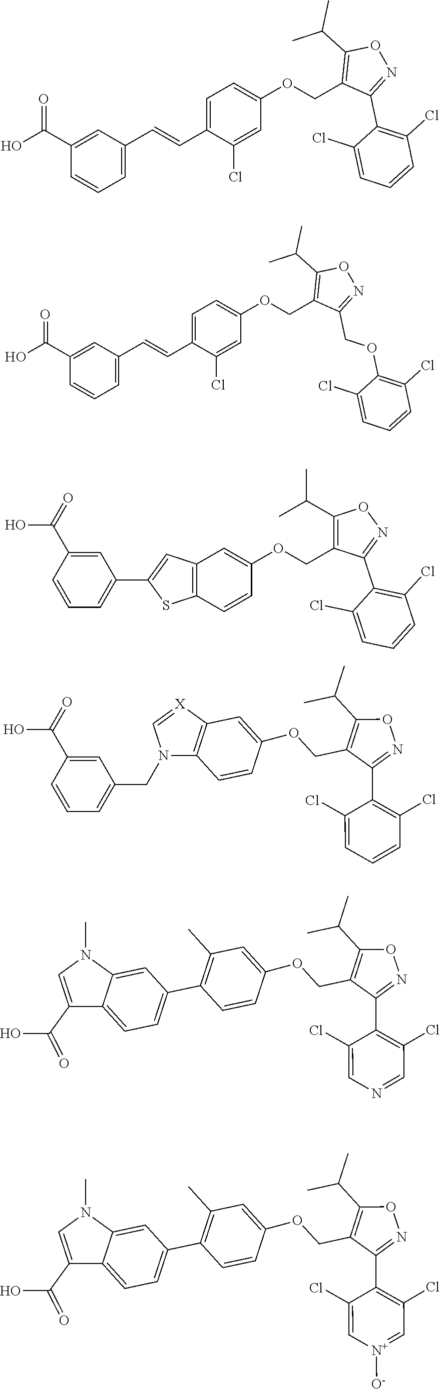

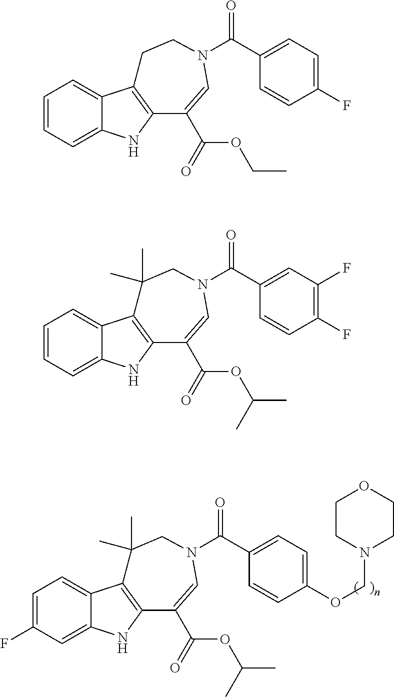

where n is 2 or 3

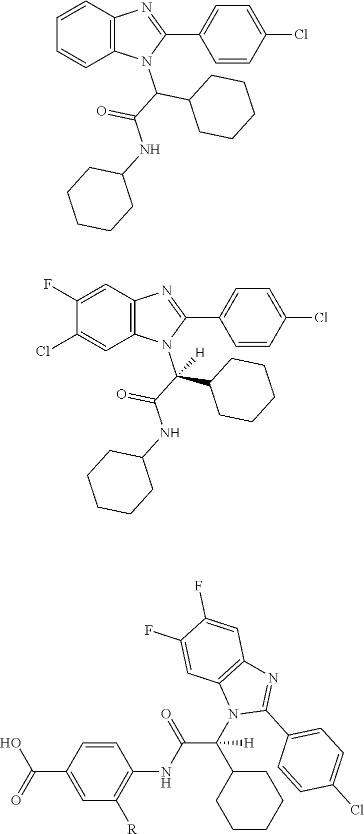

where R is H or F

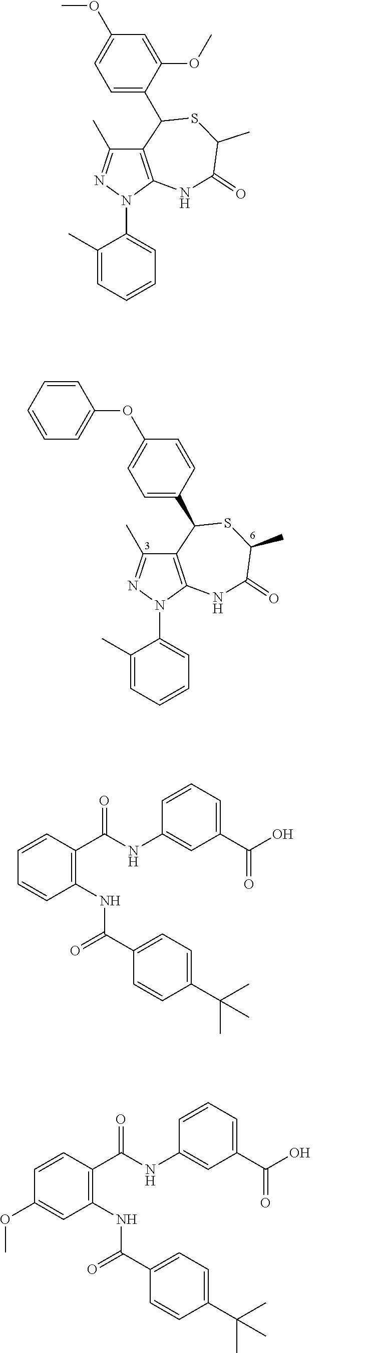

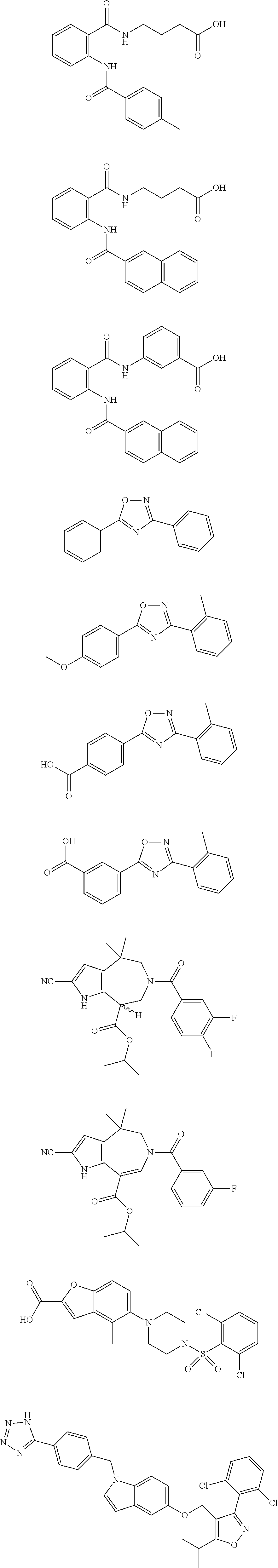

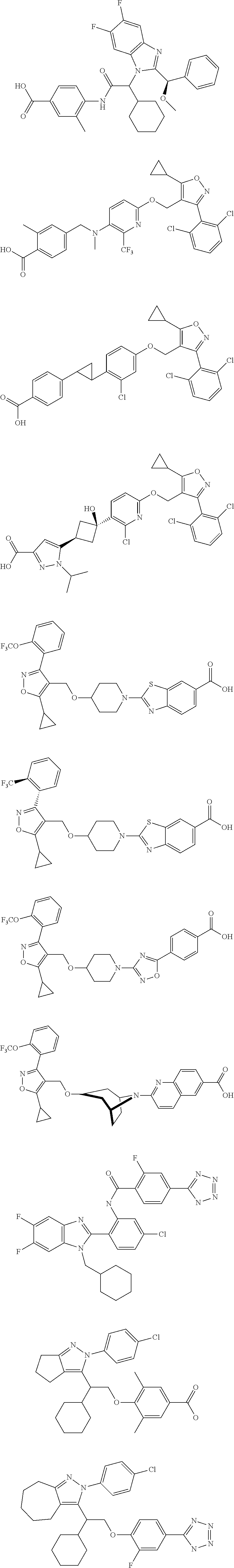

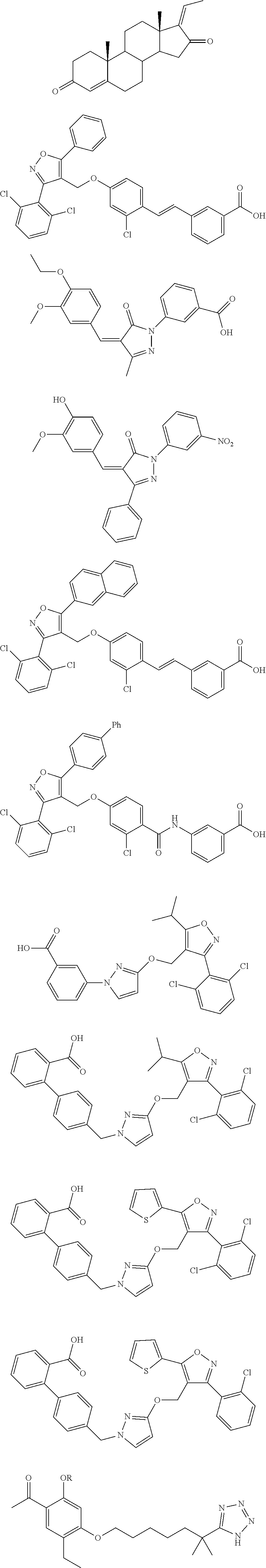

wherein R is H or benzyl,

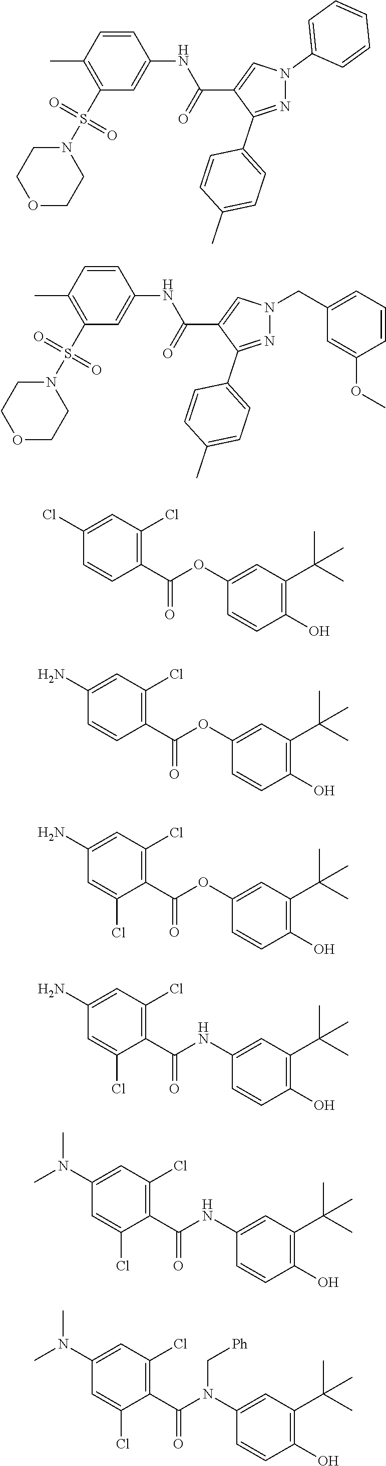

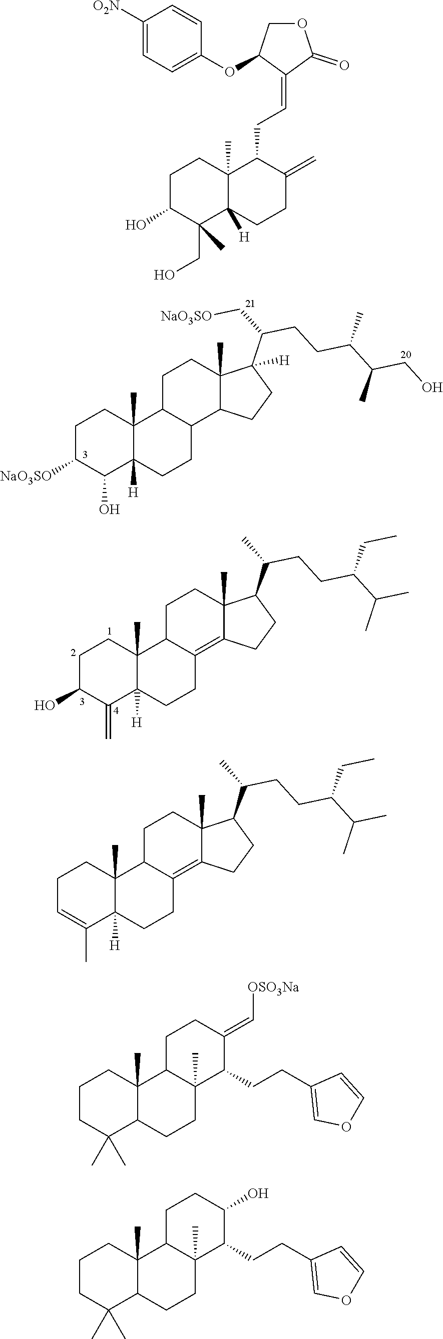

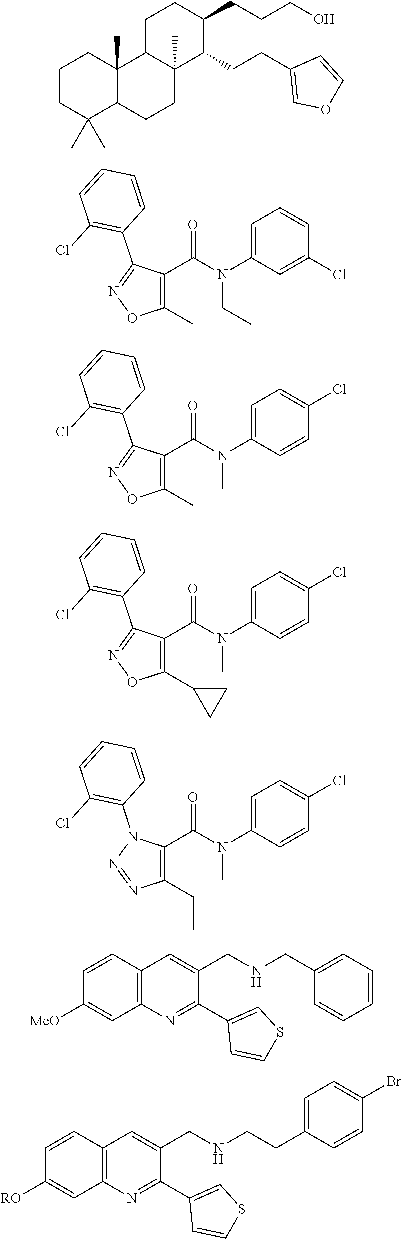

where R is H or methyl,

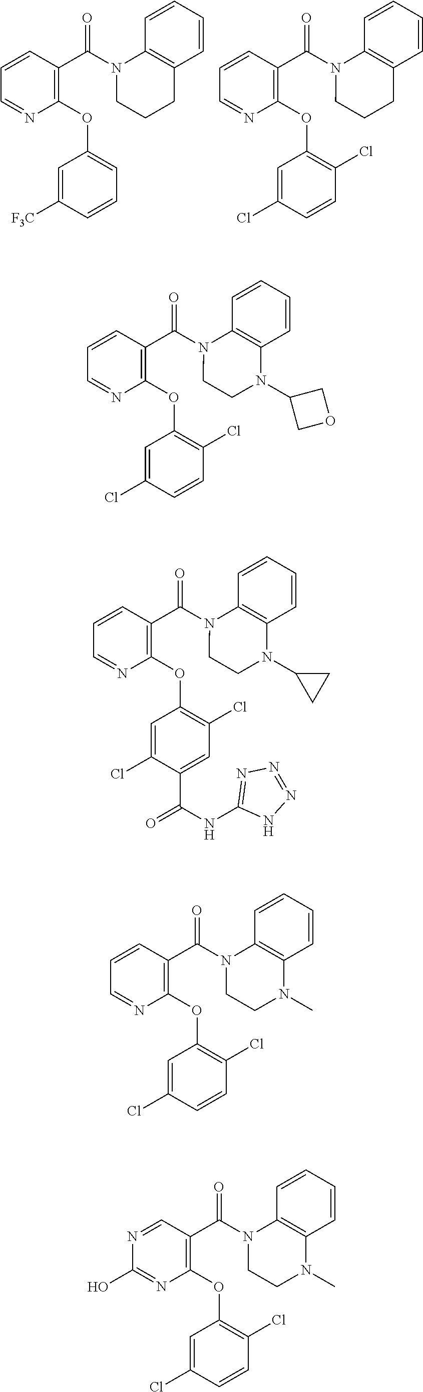

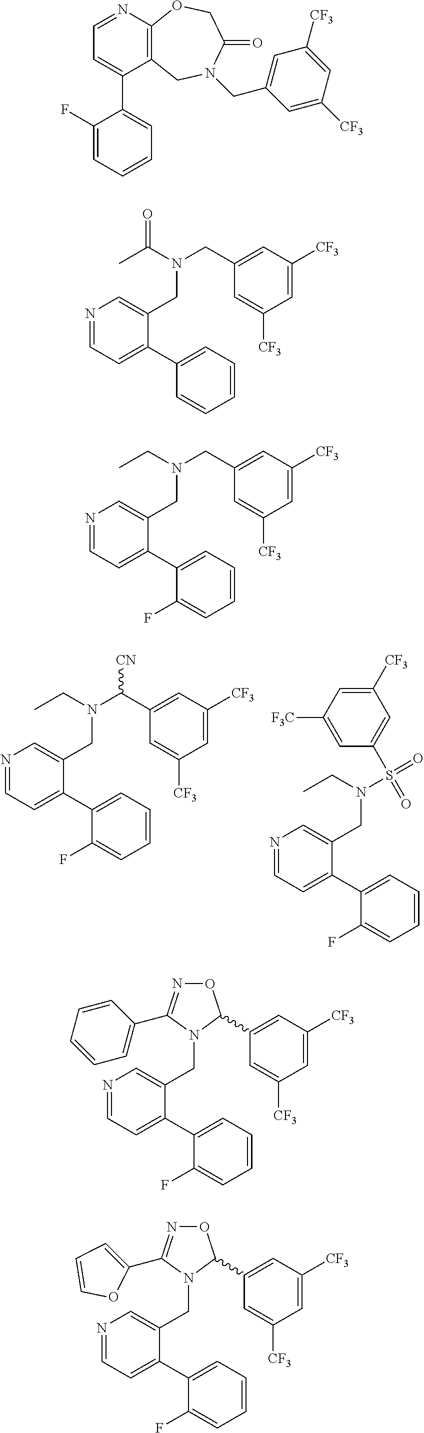

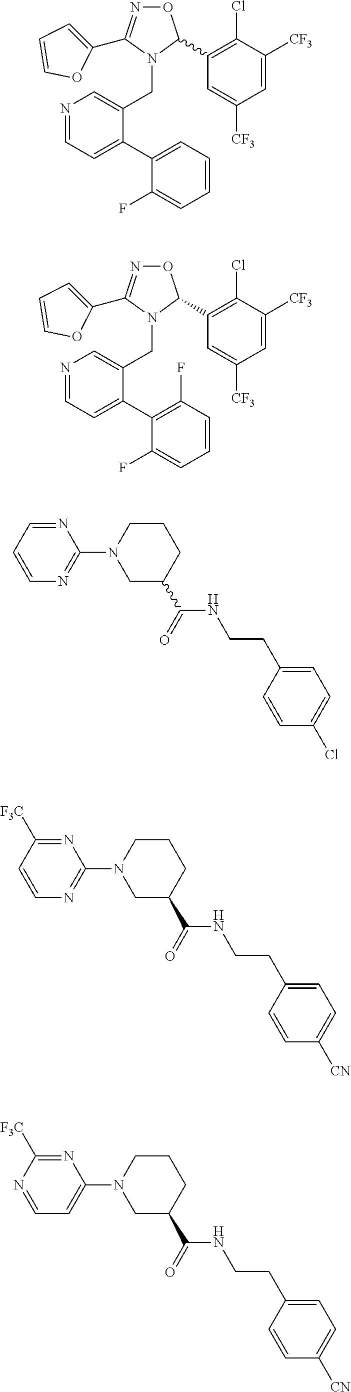

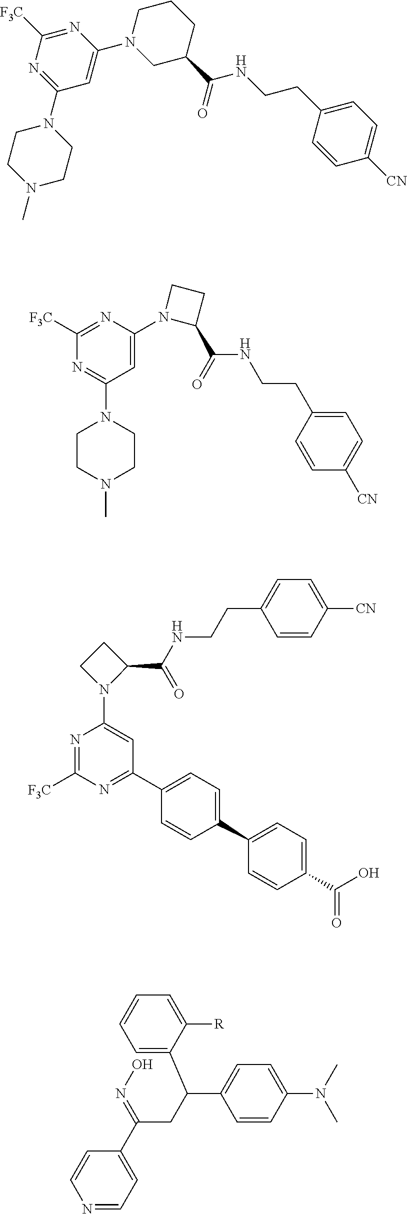

where R is H or methyl,

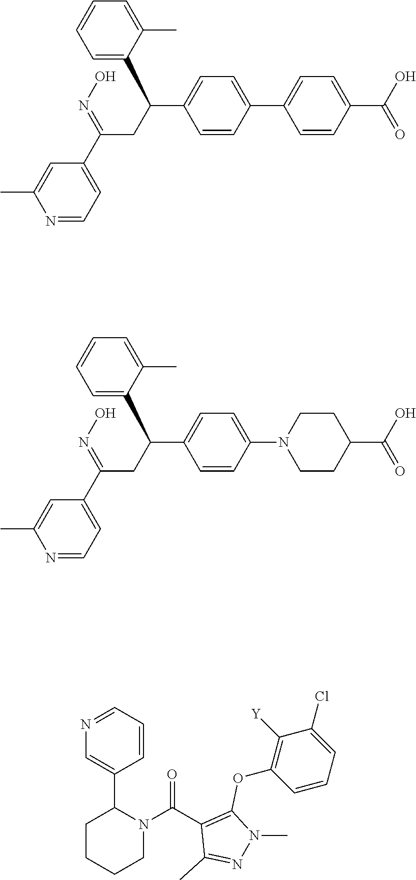

where Y is F or Cl,

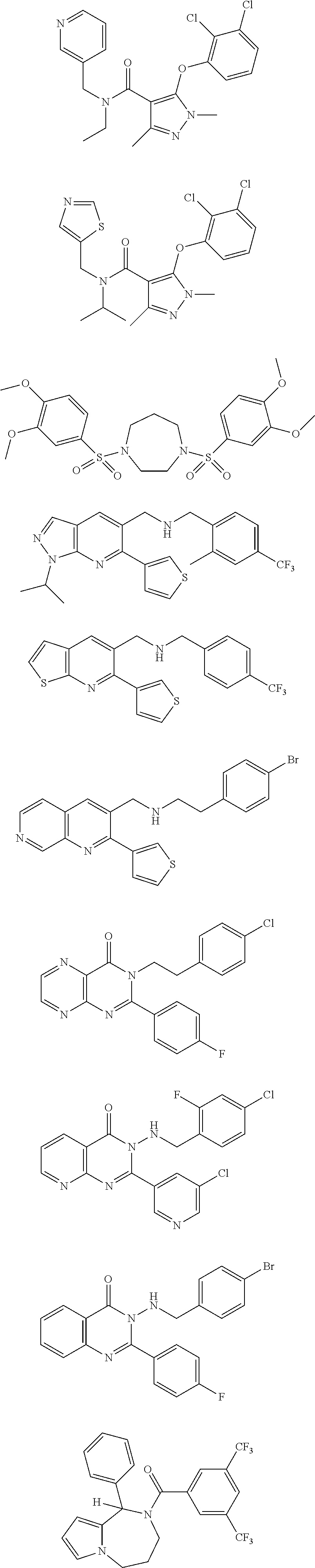

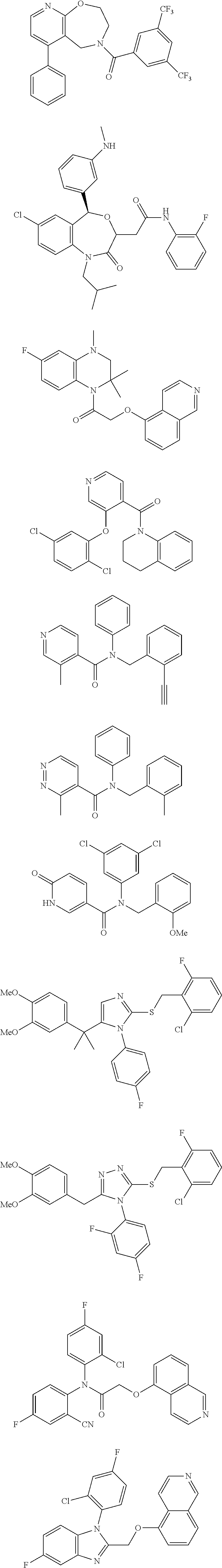

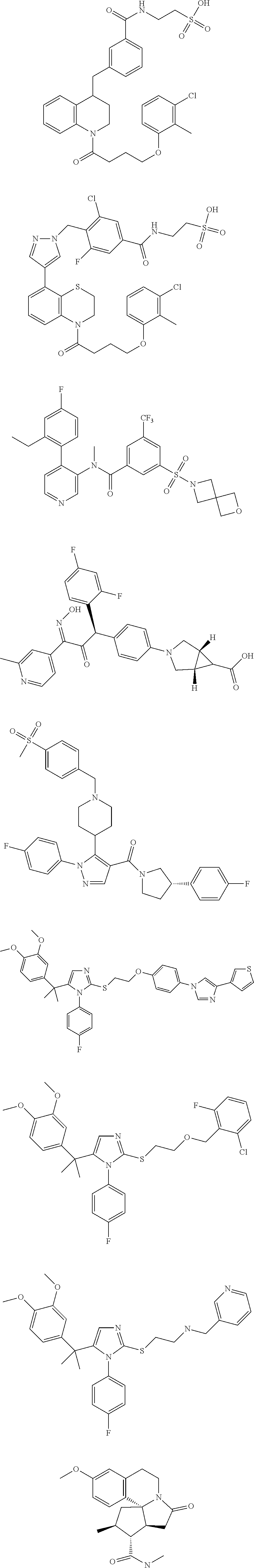

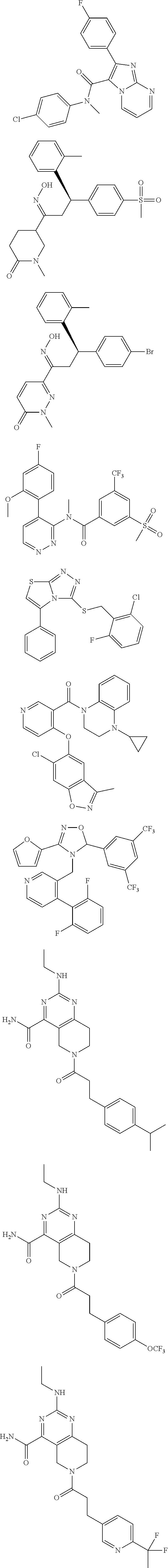

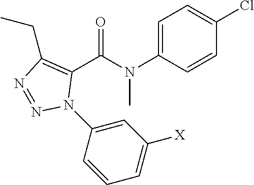

where X is H or OMe,

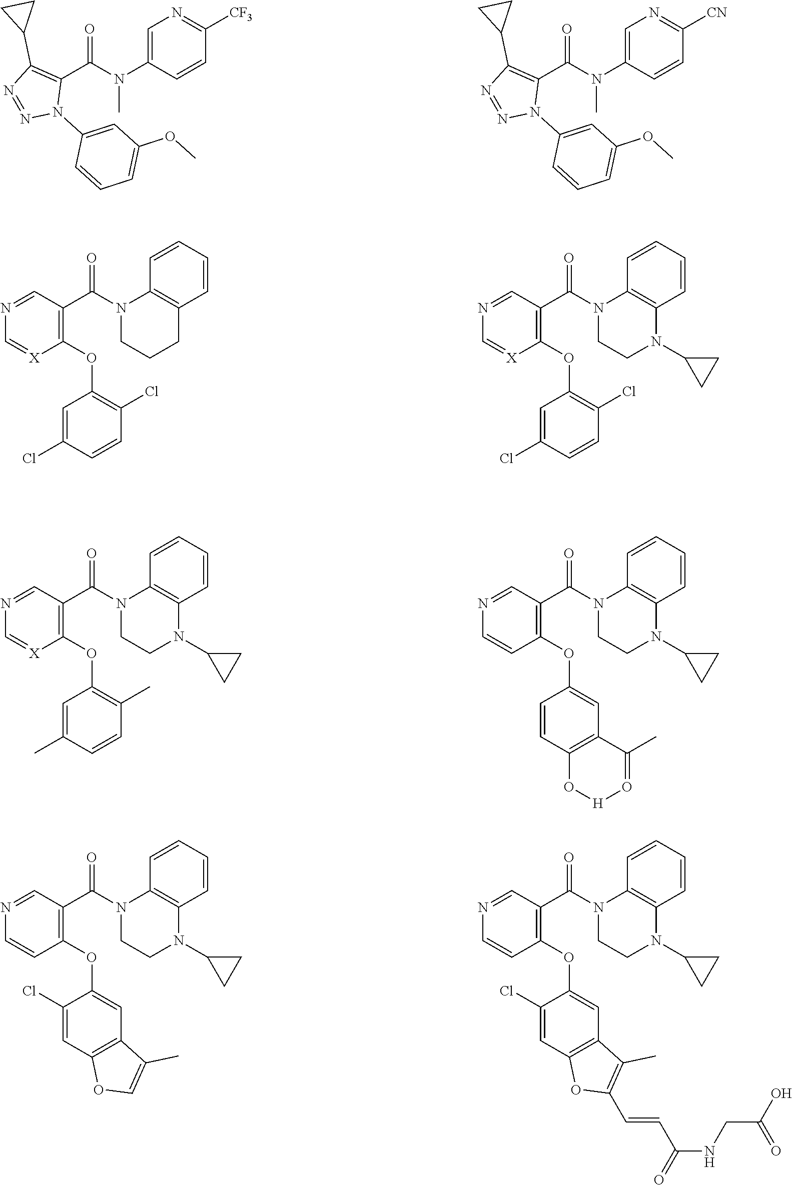

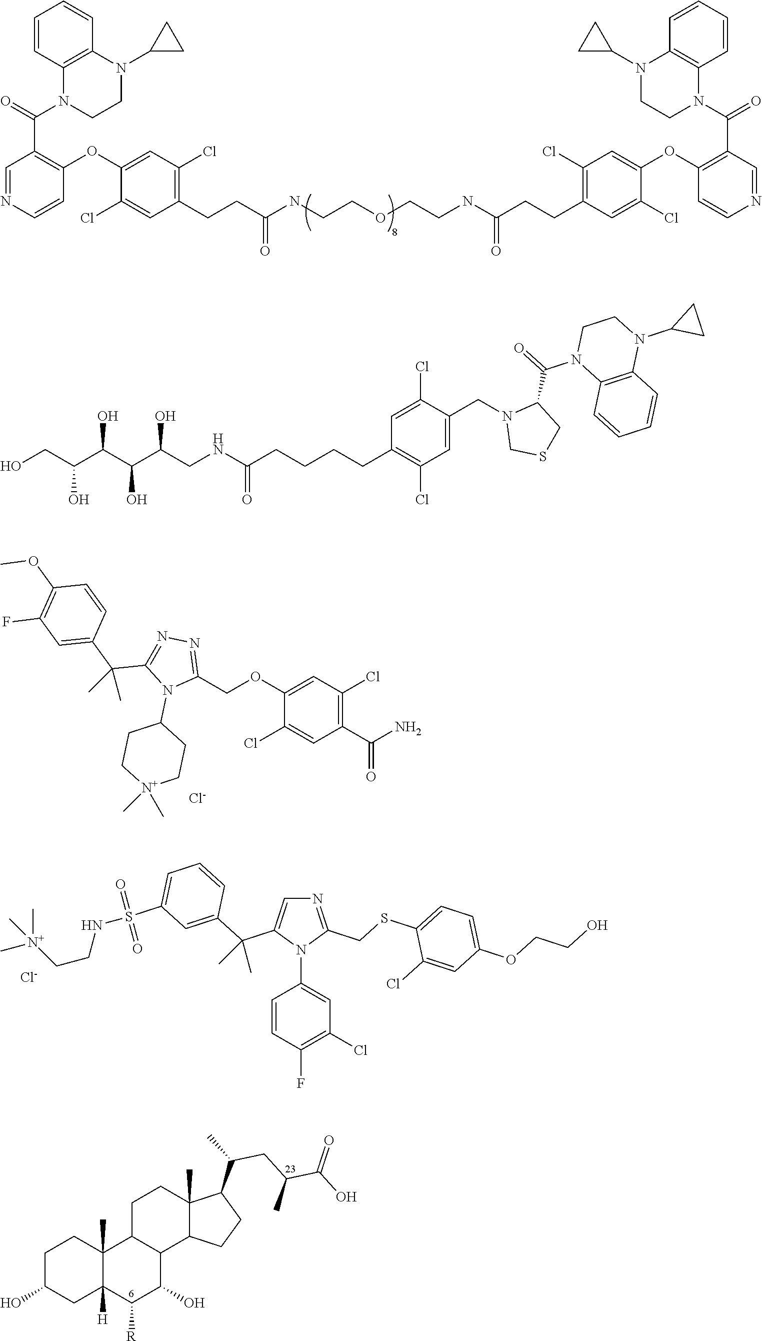

where R is H or ethyl,

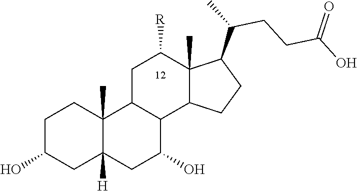

where R is H or OH,

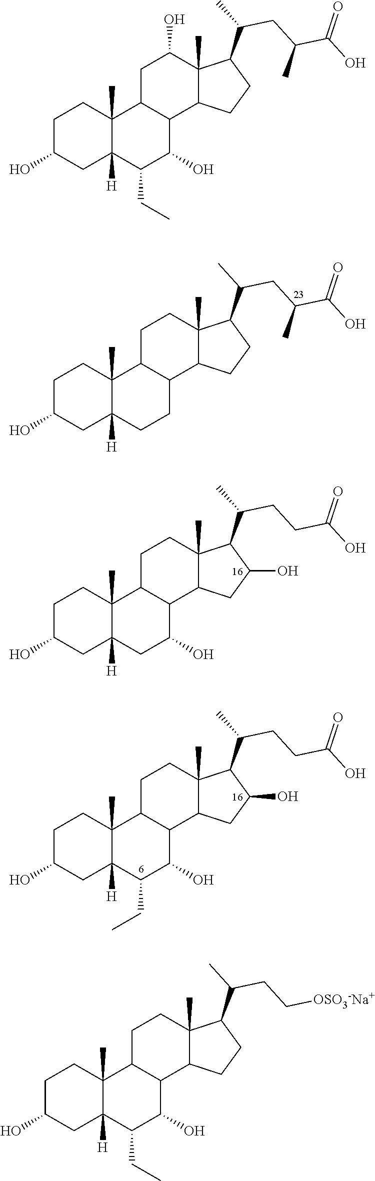

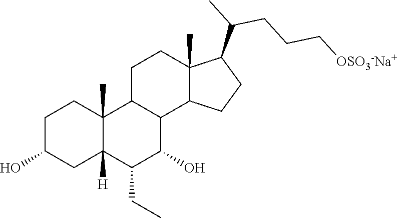

or pharmaceutically acceptable salts of any of the foregoing.

In some embodiments, the dosage forms of Compound 1 presented herein may be administered in combination with an anti-inflammatory compound. In some embodiments, the anti-inflammatory compound may be

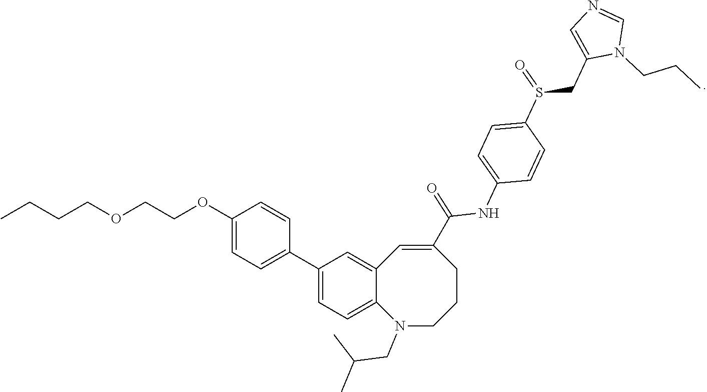 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1]

In some embodiments, the anti-inflammatory compound may be

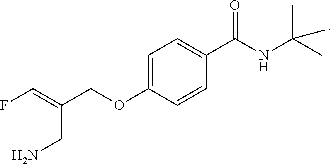 [SMILES: CC(C)(C)NC(=O)c1ccc(OC/C(=C/F)CN)cc1]

In some embodiments, the anti-inflammatory compound may be a poly-clonal or mono-clonal anti-LPS immunoglobulin. In some embodiments, the anti-LPS immunoglobulin may be IMM-124E. In some embodiments, the anti-inflammatory compound may be a pharmaceutically acceptable salt or prodrug of any of the foregoing.

In some embodiments, the second pharmaceutical agent may be an anti-fibrotic compound. In some embodiments, the anti-fibrotic compound may be

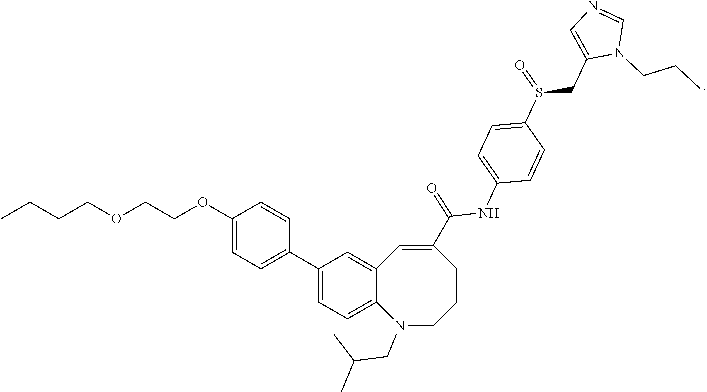 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1]

In some embodiments, the anti-fibrotic compound may be

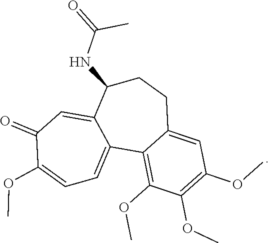 [SMILES: COc1cc2c(c(OC)c1OC)-c1ccc(OC)c(=O)cc1[C@@H](NC(C)=O)CC2]

In some embodiments, the anti-fibrotic compound may be a pharmaceutically acceptable salt or prodrug of any of the foregoing.

In some embodiments, the dosage forms of Compound 1 presented herein may be administered in combination with a GLP-1 agonist. GLP-1 are pharmaceutical compounds that may be used e.g., to treat type 2 diabetes in a subject. In some embodiments, the GLP-1 agonist may be dulaglutide. In some embodiments, the GLP-1 agonist may be exenatide. In some embodiments, the GLP-1 agonist may be liraglutide. In some embodiments, the GLP-1 agonist may be albiglutide. In some embodiments, the GLP-1 agonist may be lixisenatide. In some embodiments, the GLP-1 agonist may be semaglutide. In some embodiments, the GLP-1 agonist may be insulin glargine. In some embodiments, the GLP-1 agonist is

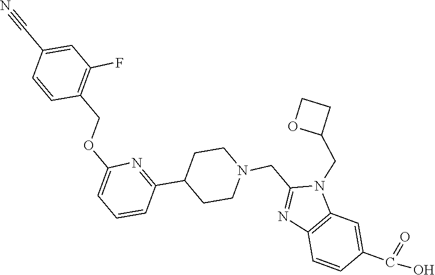 [SMILES: N#Cc1ccc(COc2cccc(C3CCN(Cc4nc5ccc(C(=O)O)cc5n4CC4CCO4)CC3)n2)c(F)c1]

(PF06882961). In some embodiments, the GLP-1 agonist may be a pharmaceutically acceptable salt or prodrug of any of the foregoing.

In some embodiments, the dosage forms of Compound 1 presented herein may be administered in combination with a GLP-1 metabolic modulator. In some embodiments, the metabolic modulator may be a thyroid hormone receptor agonist. In other embodiments, the metabolic modulator may be a selective androgen receptor modulator. In some embodiments, the metabolic modulator may be a mitochondrial membrane transport protein modulator. In other embodiments, the metabolic modulator may be a selective estrogen receptor modulator. In some embodiments, the metabolic modulator may be an inhibitor of stearoyl-CoA desaturase 1 (SCD1). In some embodiments, the metabolic modulator may be an inhibitor of dipeptidyl peptidase 4 (DPP-4). In some embodiments, the metabolic modulator may be an inhibitor of sodium glucose cotransporters 1 and/or 2 (SGLT1, SGLT2, or dual SGLT1/SGLT2 inhibitors). In some embodiments, the metabolic modulator may be recombinant fibroblast growth factor 19 (FGF19) or engineered analogs, or recombinant fibroblast growth factor 21 (FGF21) or pegylated variants thereof. In some embodiments the metabolic modulator may be

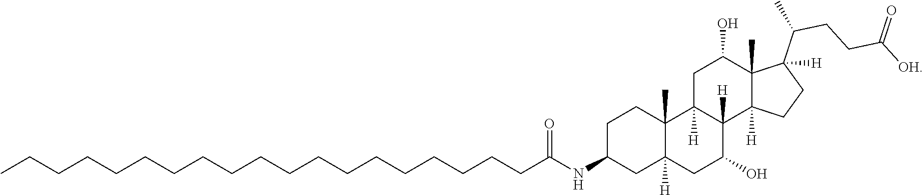

In some embodiments, the metabolic modulator may be

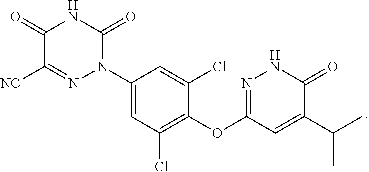 [SMILES: CC(C)c1cc(Oc2c(Cl)cc(-n3nc(C#N)c(=O)[nH]c3=O)cc2Cl)n[nH]c1=O]

In some embodiments, the metabolic modulator may be

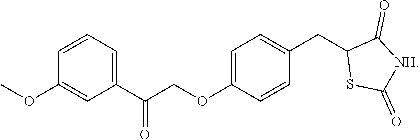 [SMILES: COc1cccc(C(=O)COc2ccc(CC3SC(=O)NC3=O)cc2)c1]

In some embodiments, the metabolic modulator may be a pharmaceutically acceptable salt or prodrug of any of the foregoing.

In some embodiments, the dosage forms of Compound 1 presented herein may be administered in combination with a fish oil derivative. Fish oils contain omega-3-fatty acids, which are polyunsaturated fatty acids (PUFAs) characterized by a double bond three atoms away from the terminal methyl group. They are widely distributed in nature and play an important role in the human diet and in human physiology, particularly with regard to lipid metabolism. In some embodiments, the fish oil derivative may be an omega-3-fatty acid alkyl ester. For example, the fish oil derivative may be an omega-3-fatty acid methyl ester, ethyl ester, n-propyl ester, or isopropyl ester. In some embodiments, the fish oil derivative may be an omega-3-fatty acid triglyceride. In some embodiments, the fish oil derivative may be ethyl (5Z,8Z,11Z,14Z,17Z)-eicosa-5,8,11,14,17-pentaenoate. In some embodiments, the fish oil derivative may be ethyl (4Z,7Z,10Z,13Z,16Z,19Z)-docosa-4,7,10,13,16,19-hexaenoate. In some embodiments, the fish oil derivative may be ethyl (7Z,10Z,13Z,16Z,19Z-docosapentaenoate. In some embodiments, the fish oil derivative may be ethyl hexadecatrienoate. In some embodiments, the fish oil derivative may be α-linolenic acid ethyl ester. In some embodiments, the fish oil derivative may be ethyl (6Z,9Z,12Z,15Z)-6,9,12,15-octadecatetraenoate. In some embodiments, the fish oil derivative may be ethyl eicosatrienoate. In some embodiments, the fish oil derivative may be ethyl eicosatetraenoate. In some embodiments, the fish oil derivative may be ethyl heneicosapentaenoate. In some embodiments, the fish oil derivative may be ethyl icosapentaenoate. In some embodiments, the fish oil derivative may be ethyl heneicosapentaenoate. In some embodiments, the fish oil derivative may be ethyl tetracosapentaenoate. In some embodiments, the fish oil derivative may be nisinic acid ethyl ester. In some embodiments, the fish oil derivative may be a pharmaceutically acceptable salt or prodrug of any of the foregoing.

### Methods of Administration

In some embodiments, the composition may be administered one, twice, three times, our four times per day. In other embodiments, the composition may be administered once, twice, or three times per week. In other embodiments, the composition is administered every other day, every three days, or every four days. In other embodiments, the composition every other week, every three weeks, or every four weeks. In other embodiments, the composition is administered once per month or twice per month.

In some embodiments, an initial loading dose is administered which is higher than subsequent doses (maintenance doses). The dosage form or mode of administration of a maintenance dose may be different from that used for the loading dose. In any of the embodiments disclosed herein, a maintenance dose may comprise administration of the unit dosage form on any dosing schedule contemplated herein, including but not limited to, monthly or multiple times per month, biweekly or multiple times each two weeks, weekly or multiple times per week, daily or multiple times per day. It is contemplated within the present disclosure that dosing holidays may be incorporated into the dosing period of the maintenance dose. Such dosing holidays may occur immediately after the administration of the loading dose or at any time during the period of administration of the maintenance dose. In some embodiments, the loading dose is 300 mg or less, 250 mg or less, 200 mg or less, 150 mg or less, 100 mg or less, 50 mg or less, 25 mg or less, 20 mg or less, 15 mg or less, 10 mg or less, or 5 mg or less. In some embodiments, the loading dose is 300 mg, 250 mg, 200 mg, 150 mg, 100 mg, 50 mg, 25 mg, 20 mg, 15 mg, 10 mg, 5, mg, or 2 mg. In some embodiments, the maintenance dose is 300 mg or less; 200 mg or less, 100 mg or less, 50 mg or less, 25 mg or less, 20 mg or less, 15 mg or less, 10 mg or less, 5 mg or less, 2.5 mg or less, or 1 mg or less. In some embodiments, the maintenance dose is 300 mg, 250 mg, 200 mg, 100 mg, 50 mg, 25 mg, 20 mg, 15 mg, 10 mg, 5 mg, 2.5 mg, or 1 mg.

### Methods of Treatment

Some embodiments according to the methods and compositions of the present disclosure relate to a method for preventing, treating, or ameliorating one or more fatty liver diseases in a subject comprising administering an effective amount of a Compound 1 described herein in combination with one or more second pharmaceutical agents to a subject in need thereof. In some embodiments, the fatty liver disease may be steatosis. In other embodiments, the fatty liver disease may be non-alcoholic fatty liver disease. In some embodiments, the fatty liver disease may be non-alcoholic steatohepatitis (NASH). In some embodiments, the subject may have two or more of the aforementioned fatty liver diseases.

Some embodiments according to the methods and compositions of the present disclosure relate to a method for the reduction or prevention of the deposition of extracellular matrix proteins, comprising administering an effective amount of Compound 1 described herein in combination with one or more second pharmaceutical agents described herein to a subject in need thereof. In some embodiments, said deposition of extracellular matrix proteins may comprise abnormal or excessive deposition of said proteins. In some embodiments, said extracellular matrix proteins may comprise one or more of collagen, keratin, elastin, or fibrin. In some embodiments, said extracellular matrix proteins may comprise collagen. In some embodiments, said extracellular matrix proteins may comprise Type I collagen. In some embodiments, said extracellular matrix proteins may comprise Collagen Type Ia. In some embodiments, said extracellular matrix proteins may comprise Type III collagen. Some embodiments according to the compositions and methods of the present disclosure relate to a method for the treatment of a fibrosis or its symptoms or sequelae, comprising administering an effective amount of a compound described herein to a subject in need thereof.

In some embodiments, the compounds and compositions comprising Compound 1 described herein and/or one or more second pharmaceutical agents described herein can be used to treat a variety of conditions arising from fibrosis or inflammation, and specifically including those associated with abnormal collagen deposition. Example conditions include glycogen storage disease type III (GSD Ill), glycogen storage disease type VI (GSD VI), glycogen storage disease type IX (GSD IX), non-alcoholic steatohepatitis (NASH), cirrhosis, hepatitis, scleroderma, alcoholic fatty liver disease, atherosclerosis, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, pulmonary fibrosis, idiopathic pulmonary fibrosis, diffuse parenchymal lung disease, idiopathic interstitial fibrosis, diffuse interstitial fibrosis, interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-polymyositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis, or symptoms or sequelae thereof, or other diseases or conditions resulting in the excessive deposition of extracellular matrix components, such as collagen.

In some embodiments the methods of the present disclosure comprise methods for the treatment, amelioration, or prevention of a fibrotic condition. In some embodiments, said fibrotic condition may be secondary to another condition. In some embodiments, said fibrotic condition or primary condition may further comprise chronic inflammation of an organ, tissue, spatial region, or fluid-connected area of the body of a subject. In some embodiments, said inflammation may comprise activation of one or more TGF-beta dependent signaling pathways. In some embodiments, said TGF-β dependent signaling pathways may comprise one or more elements responsive to T3 or T4. In some embodiments, said fibrotic condition may comprise abnormal or excessive deposition of one or more of collagen, keratin, or elastin. In some embodiments, said fibrotic condition may comprise abnormal or excessive deposition of collagen. In some embodiments, said fibrotic condition may comprise abnormal or excessive deposition of Type I collagen. In some embodiments, said fibrotic condition may comprise abnormal or excessive deposition of Collagen Type Ia. In some embodiments, said fibrotic condition may comprise abnormal or excessive deposition of Type III collagen. In some embodiments said fibrotic condition may comprise one or more of glycogen storage disease type III (GSD III), glycogen storage disease type VI (GSD VI), glycogen storage disease type IX (GSD IX), non-alcoholic steatohepatitis (NASH), cirrhosis, hepatitis, scleroderma, alcoholic fatty liver disease, atherosclerosis, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, scleroderma, pulmonary fibrosis, idiopathic pulmonary fibrosis, diffuse parenchymal lung disease, idiopathic interstitial fibrosis, diffuse interstitial fibrosis, interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-polymyositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis. In some embodiments, said fibrotic condition may comprise one or more of GSD III, GSD IX, Non Alcoholic Steatohepatitis, cirrhosis of the liver and/or pancreas, scleroderma, idiopathic pulmonary fibrosis, psoriasis, alcoholic fatty liver disease, Dupuytren's disease, and/or any combination thereof.

According to the methods and composition as disclosed herein, the fibrotic condition or condition having fibrosis as a sequela may further comprise chronic inflammation. According to the methods and compositions as disclosed herein, the fibrotic condition or condition having fibrosis as a sequela may further comprise activation of one or more TGF-β dependent signaling pathways. According to the methods and compositions as disclosed herein, the fibrotic condition or condition having fibrosis as a sequela may further comprise activation and/or repression of one or more Thyroid Receptor Beta (TRβ) dependent signaling pathways. According to the methods and compositions as disclosed herein, the fibrotic condition or condition having fibrosis as a sequela may further comprise the involvement of signaling pathways responsive to triiodothyronine (T3), thyroxine (T4), any combination thereof, or mimetics thereof. According to the methods and compositions as disclosed herein, the fibrotic condition or condition having fibrosis as a sequela may further comprise the involvement of receptors responsive to T3, T4, any combination thereof, or mimetics thereof. In some embodiments according to the methods and compositions disclosed herein, the fibrotic condition or condition having fibrosis as a sequela may comprise the involvement of TRβ.

In some embodiments, the compositions and methods described herein provide compositions and methods for the treatment, amelioration, prevention or cure of collagen deposition. In some embodiments, said collagen deposition comprises and abnormal or excessive deposition of collagen. In some embodiments, said collagen deposition may comprise abnormal or excessive deposition of Type I collagen. In some embodiments, said collagen deposition may comprise abnormal or excessive deposition of Collagen Type Ia. In some embodiments, said collagen deposition may comprise abnormal or excessive deposition of Type III collagen. According to the methods and compositions as disclosed herein, said collagen deposition may further comprise the involvement of receptors responsive to T3, T4, any combination thereof, or mimetics thereof. In some embodiments according to the methods and compositions disclosed herein, said collagen deposition may comprise the involvement of TRβ. In some embodiments according to the methods and compositions disclosed herein, said collagen deposition may be prevented, ameliorated, or cured by the administration of one or more agonists of TRβ.

In some embodiments, administration of dosage forms of Compound 1 described herein results in a reduction in the expression of the Cola1, Col3a1, αSMA, and/or Galectin1 genes or any combination or product thereof in the subject to which said combination is administered. In some embodiments, administration of dosage forms of Compound 1 described herein results in a reduction in the degree of fibrosis observable by histology, histochemistry, immunohistochemistry, or the like, and/or reductions in the amount, accumulation, or distribution of type 1 collagen and/or hydroxyproline or any combination thereof in the subject to which said combination is administered. In some embodiments, administration of dosage forms of Compound 1 described herein as disclosed herein results in a reduction in total serum lipids, total serum cholesterol, total serum triglycerides, total liver lipids, total liver cholesterol, total liver triglycerides, or any combination thereof.

### EXAMPLES

The compositions and methods described herein are further illustrated by the following non-limiting examples.

### Example 1: Preparation of Spray-Dried Dispersions of Compound 1

The following formulations were prepared and spray dried to form amorphous spray dried dispersions (SDD) for further testing: (a) 25/75 (w/w) Compound 1/hydroxypropylmethylcellulose acetate succinate type M (HPMCAS-M); (b) 25/75 (w/w) Compound 1/hydroxypropylmethylcellulose acetate succinate type L (HPMCAS-L); (c) 25/75 (w/w) Compound 1/polyvinylpyrrolidinone-vinyl acetate copolymer (PVP-VA64); (d) 25/75 (w/w) Compound 1/Eudragit® Li100; and (e) (w/w) Compound 1/HPMC E3.

These SDD intermediate formulations were spray dried on the Bend Lab Dryer with 35 kg/h drying gas flow rate capacity (BLD-35). The BLD-35 is a custom-built spray dryer to support batch sizes from 0.5 to <1 kg batch sizes for early feasibility, pharmacokinetic, and toxicology supplies. The BLD-35 uses a pressure swirl nozzle to atomize the spray drying solution into droplets, which are then dried with a heated nitrogen stream and collected using a cyclone. The manufacturing conditions are provided in Table 1. All five SDD were manufactured successfully with high yields. While a small amount of insoluble particles were noted in the acetone solution, they did not affect the process and are believed to be impurities or other non-Compound 1 materials. The spray dried dispersions exhibited typical SDD morphology showed no evidence of crystallinity

TABLE 1
Manufacturing Conditions for Spray Dried Dispersions of Compound 1

| PARAMETER | VALUE | | | | |
|---|---|---|---|---|---|
| Formulation | 25/75 | 25/75 | 25/75 | 25/75 | 25/75 |
| | Compound | Compound | Compound | Compound | Compound |
| | 1/HPMCAS-L | 1/HPMCAS-M | 1/PVP-VA64 | 1/Eudragit | 1/HPMC E3 |
| | | | | L100 | |
| Lot Number | BREC- | BREC- | BREC- | BREC- | BREC- |
| | 2249-002A | 2249-002B | 02249-002C | 00249-002D | 2249-002E |

| Process Date | 27 Mar. 2019 | | |
|---|---|---|---|
| Spray Dryer | BLD-35 (BRI-EQ-0036) | | |
| Solvent | Acetone | ~97/3 (wt) Acetone/Water | 8/2 (wt) Acetone/Water |

| Solids | 6 | | | | |
|---|---|---|---|---|---|
| Loading (wt %) | | | | | |
| Batch Size (g) | 12 | | | | |
| Nozzle | Schlick 2.0 (0.2 mm orifice) [C-1037] | | | | |
| Atomization | 120 | | | | |
| Pressure (psig) | | | | | |
| Solution Feed | ~33 | ~29 | ~35 | ~23 | ~29 |
| Rate (g/min) | | | | | |
| Drying Gas Flow | ~500 | | | | |
| Rate (g/min) | | | | | |
| Inlet Gas | 100 | 98 | 93 | 97 | 100 |
| Temperature (° C.) | | | | | |
| Outlet Gas | 43 | 42 | 42 | 43 | 43 |
| Temperature (° C.) | | | | | |
| Yield (%) | 95 | 96 | 91 | 97 | 95 |

### Example 2: Aqueous Solubility Compound 1

The equilibrium solubility of crystalline Compound 1 in 0.01N HCl and PBS buffer at pH 6.5 at 37° C. was assessed by measuring the dissolved concentration of Compound 1 from saturated solutions in each respective media. Experimental values for solubility, listed in Table 2, were obtained by measuring supernatant concentrations after centrifugation at 386,000×g.

TABLE 2
Solubility of Amorphous Compound 1

Ultracentrifuge Concentration
(μg Compound 1/mL

| Media | 90 min | 20 hours |
|---|---|---|
| 0.01N HCl | <0.05 | <0.05 |
| PBS, pH 6.5 | <0.05 | 0.6 |
| 0.5% Simulated intestinal | 92 | 87 |
| fluid (SIF) in PBS, pH 6.5 | | |

The amorphous solubility of amorphous Compound 1 was determined by slow addition of the API from an organic stock solution into the aqueous medium of interest. When the amorphous solubility was reached, a drug rich phase formed and was measured by detection by scattering of UV/Visible light and/or by dynamic light scattering (DLS).

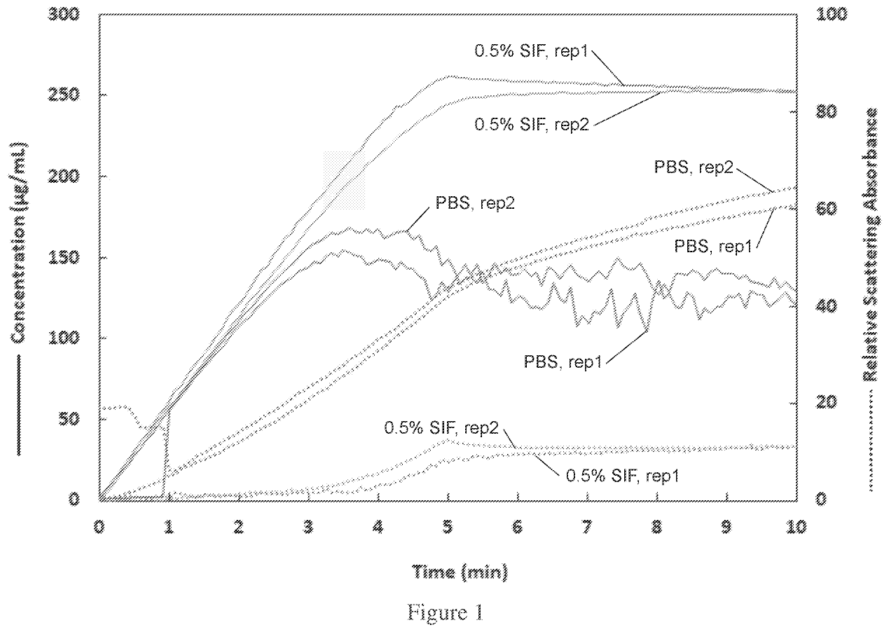

The solubility of amorphous Compound 1 was evaluated in PBS at pH 6.5 alone and with 0.5% simulated intestinal fluid (SIF). FIG. 1 shows the measured concentration of Compound 1 from a stock solution in methanol, as well as light scattering over time. The onset of scattering in the SIF containing media occurred after 180-210 μg/mL Compound 1. In PBS alone, Compound 1 precipitated as soon as the stock solution was introduced to the medium, so an estimate for the amorphous solubility was not obtained. This is consistent with the initial solubility testing where the crystalline solubility in PBS in the absence of bile salts is exceedingly low.

### Example 3: Solubility of Spray Dried Dispersions of Compound 1

The SDDs were evaluated in a non-sink dissolution test designed to monitor dissolution rate, as well as inhibition of crystallization, on transfer from simulated gastric to simulated intestinal media. For the gastric-to-intestinal buffer (G-IB) transfer dissolution test, pH 2 (“gastric”; “G”) media was added to each sample at a target dose concentration of 1000 μg Compound 1/mL. After 30 minutes of exposure to gastric media, concentrated simulated intestinal buffer was added to reach a final composition of 0.5% SIF in PBS, pH 6.5 at half the dose concentration (500 μg Compound 1/mL). This dose concentration was selected at twice the estimated amorphous solubility in an attempt to discriminate on dissolution rate and sustainment of supersaturation simultaneously. The concentration was monitored by UV fiber optic probes in situ (stir rate: 100 revolutions per minute) as well as being assessed by either microcentrifugation (15,800×g) or ultracentrifugation (300,000×g). The microcentrifuge separated the precipitate (undissolved drug) and total solubilized drug. Total solubilized drug consists of three solubilized species: freely solubilized drug (“free drug”), drug associated with bile salt micelles (“micelle bound drug”), and drug in small aggregate roughly 50-300 nm in size (“colloidal drug”). The ultracentrifuge further separates the colloidal drug and the supernatant contains only free drug and micelle bound drug. These three species have different activities in vivo and can be used to help differentiate formulations. Free drug is the smallest species and is the only species considered to partition directly into the cell membrane of the epithelium. Micelle bound drug can cross the unstirred mucous boundary layer and source free drug once a concentration gradient has been created by freely solubilized drug being absorbed. Finally, the drug-polymer colloids are a source of rapidly dissolving drug and may, in some cases, penetrate the unstirred mucous boundary layer (Stewart et al. Mol. Pharmaceutics, 2017, 12, 2437-2449)

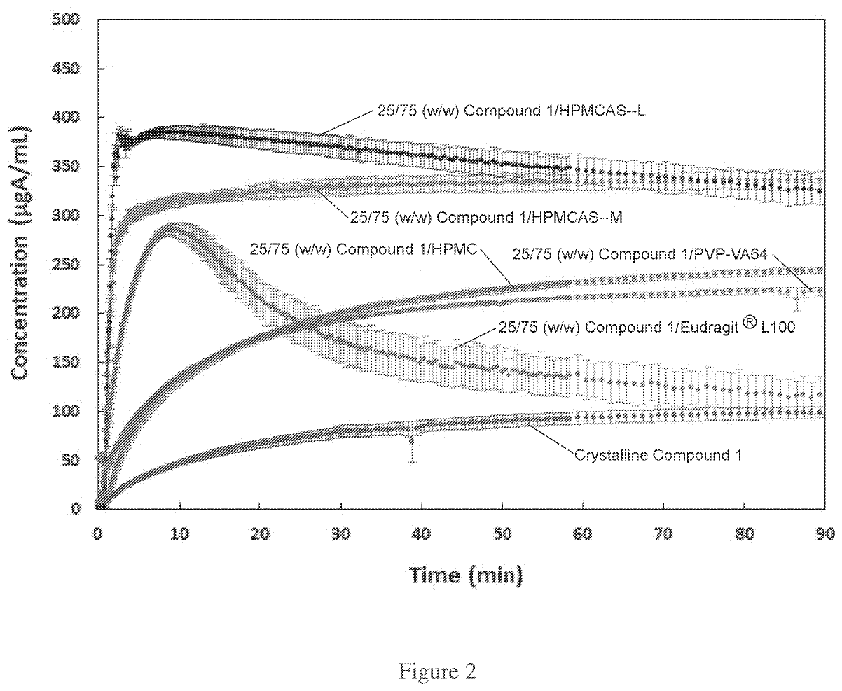

The results of the G-IB dissolution test are shown in FIG. 2 and Table 3. All SDD formulations containing enteric polymers reached the amorphous solubility in intestinal buffer within 10 minutes. These dispersions also appear to form colloids in IB with the 25/75 Compound 1/HPMCAS-L SDD affording the highest concentration. The in situ measurements for the Eudragit containing SDD did not agree with the concentrations measured after centrifugation. A closer look at the raw data from the UV probes showed a spectral shift for this sample that led to the erroneous measurement. The exact reason for this shift is currently unknown. It may be attributable to detector saturation, arising from the undissolved solids, or a specific interaction between the drug and polymer in solution. All SDDs exhibited a significant improved dissolution as compared to crystalline Compound 1.

TABLE 3
G-IB Dissolution of Spray Dried Dispersions of Compound 1

| | Concentration (μg Compound 1/mL | | | | |
|---|---|---|---|---|---|
| | G 20 min | IB 10 min | | IB 90 min | |
| Sample | micro | micro | ultra | micro | ultra |
| 25/75 (w/w) Compound | 18 | 277 | 251 | 329 | 254 |
| 1/HPMCAS-M SDD | | | | | |
| 25/75 (w/w) Compound | 7 | 407 | 251 | 420 | 252 |
| 1/HPMCAS-L SDD | | | | | |
| 25/75 (w/w) Compound | 4 | 156 | 191 | 228 | 226 |
| 1/PVP-VA64 SDD | | | | | |
| 25/75 (w/w) Compound | 15 | 330 | 262 | 319 | 256 |
| 1/Eudragit ® L100 SDD | | | | | |
| 25/75 (w/w) Compound | 30 | 148 | 168 | 240 | 239 |
| 1/HPMC E3 SDD | | | | | |
| Crystalline Compound 1 | 0 | 39 | 49 | 88 | 88 |

### Example 4: Open Stability of Spray Dried Dispersions of Compound 1

SDDs of Compound 1 were aged at 40° C./75% Relative Humidity (RH) open for 2 weeks to provide an initial assessment of Compound 1 SDD chemical stability. The stability samples were tested using H-PLC using the method in Table 4.

TABLE 4
HPLC Method for Stability Analysis

| Working Concentration | ~100 | μgA/mL |
|---|---|---|
| MPA (35%) | 20 mM Sodium Phosphate (aq), pH 6.1 | |
| MPB (65%) | ACN | |
| Diluent | 7:3 ACN:20 mM Sodium Phosphate buffer | |
| Column | XBridge Shield RP18, 5 μm, 4.6 × 50 mm | |
| Column Temp | 30° | C. |
| Detection Wavelength | 280 | nm |
| Flow Rate | 1 | mL/min |
| Injection Volume | 10 | μL |

The stability samples were tested for assay and related substances. Results of the analysis are listed in Table 5. Possible degradation from processing was observed for all SDD formulations, with the increase in total impurities ranging from 0.65% (HPMCAS-M SDD) to 1.42% (HPMCAS-L SDD). This is mainly due to an API process-related impurity.

TABLE 5
HPLC Open Stability Data for Spray
Dried Dispersions of Compound 1

| | | Compound 1 | Impurities |
|---|---|---|---|
| | | Relative | Relative |
| | | abundance | abundance |
| | | (%) | (%) |
| Compound 1 | Stored at Room Temp. | 97.61 | 2.47 |
| 25/75 (w/w) | Stored at 5° C. | 96.88 | 3.12 |
| Compound | 2 weeks at 40° C. | 96.33 | 3.69 |
| 1/HPMCAS-M | and 75% RH open | | |
| 25/75 (w/w) | Stored at 5° C. | 96.19 | 3.89 |
| Compound | 2 weeks at 40° C. | 95.68 | 4.44 |
| 1/HPMCAS-L | and 75% RH open | | |
| 25/75 (w/w) | Stored at 5° C. | 96.87 | 3.18 |
| Compound | 2 weeks at 40° C. | 96.78 | 3.25 |
| 1/PVP-VA64 | and 75% RH open | | |
| 25/75 (w/w) | Stored at 5° C. | 96.53 | 3.54 |
| Compound | 2 weeks at 40° C. | 95.47 | 4.56 |
| 1/Eudragit ® | and 75% RH open | | |
| L100 | | | |
| 25/75 (w/w) | Stored at 5° C. | 96.59 | 3.46 |
| Compound | 2 weeks at 40° C. | 94.20 | 5.80 |
| 1/HPMC E3 | and 75% RH open | | |

The PVP-VA containing dispersion exhibited the best chemical stability at 40° C. and 75% RH. The neutral PVP-VA polymer may well be the least reactive with Compound 1. The Eudragit and HPMC containing dispersions had the worst chemical stability at 40° C. and 75% RH. This is mostly due to growth of hydrolytic degradation products of Compound 1 in the Eudragit containing SDD and due to growth of unknown degradants in the HPMC containing SDD. Similar growth of total impurities was observed for the HPMCAS-M and HPMCAS-L containing SDDs after 2 weeks at 40° C. and 75% Relative Humidity open.

### Example 5: Closed Stability Packaging Study of Compound 1 Spray Dried Dispersions

Due to the extensive degradation observed after 2 weeks at 40° C./75% RH open, a closed with desiccant condition stability study was initiated for Compound 1 SDDs. Samples were evaluated for stability at 2 weeks, 6 weeks, and 3 months when stored in 4 mL scintillation vials and sealed in 60 cc HDPE bottles with heat induction sealing and 0.5 g desiccant.

Chemical stability of Compound 1 SDDs after 2 weeks under closed conditions was evaluated by HPLC using the method provided herein in Table 4. The PVP-VA containing SDD exhibited the best chemical stability after 2 weeks under closed conditions with little to no degradation observed. Among the SDDs with enteric (acidic) polymers, the HPMCAS-M based SDD provided the best chemical stability profile. All SDDs containing cellulosic polymers show the same growth of an impurity after 2 weeks at 40/75 closed. Upon closer inspection, this impurity is present in the in-going API at a level that was below the threshold used for integration. The HPMC containing dispersion exhibited the worst chemical stability under closed conditions. The Eudragit containing dispersion carries moderate risk to chemical stability at 40/75 closed with a slight increase in hydrolytic degradation products of Compound 1 as well as an unknown impurity after 2 weeks. Further evidence of unknown degradants that may form during manufacture and co-elute with known synthetic impurities was observed. Closed stability degradation data at two weeks is provided in Table 6, while closed stability degradation data at six weeks is provided in Table 7. Additionally, no changes in particle morphology and crystallinity for the SDDs after 6 weeks at 40° C./75% RH were observed.

TABLE 6
HPLC 2 Week Closed Stability Data for
Spray Dried Dispersions of Compound 1

| | | Compound 1 | Impurities |
|---|---|---|---|
| | | Relative | Relative |
| | | abundance | abundance |
| | | (%) | (%) |
| Compound 1 | Stored at Room Temp. | 97.07 | 2.95 |
| 25/75 (w/w) | Stored at 5° C. | 96.88 | 3.12 |
| Compound | 2 weeks at 25° C./60% | 96.81 | 3.21 |
| 1/HPMCAS-M | RH closed with dessicant | | |
| | 2 weeks at 40° C./75% | 96.59 | 3.43 |
| | RH closed with dessicant | | |
| 25/75 (w/w) | Stored at 5° C. | 96.19 | 3.89 |
| Compound | 2 weeks at 25° C./60% | 96.29 | 3.82 |
| 1/HPMCAS-L | RH closed with dessicant | | |
| | 2 weeks at 40° C./75% | 96.04 | 4.00 |
| | RH closed with dessicant | | |
| 25/75 (w/w) | Stored at 5° C. | 96.87 | 3.18 |
| Compound | 2 weeks at 25° C./60% | 96.85 | 3.15 |
| 1/PVP-VA64 | RH closed with dessicant | | |
| | 2 weeks at 40° C./75% | 96.82 | 3.18 |
| | RH closed with dessicant | | |
| 25/75 (w/w) | Stored at 5° C. | 96.53 | 3.54 |
| Compound | 2 weeks at 25° C./60% | 96.70 | 3.34 |
| 1/Eudragit ® | RH closed with dessicant | | |
| L100 | | | |
| | 2 weeks at 40° C./75% | 96.58 | 3.54 |
| | RH closed with dessicant | | |
| 25/75 (w/w) | Stored at 5° C. | 96.59 | 3.46 |
| Compound | 2 weeks at 25° C./60% | 96.49 | 3.51 |
| 1/HPMC E3 | RH closed with dessicant | | |
| | 2 weeks at 40° C./75% | 96.14 | 3.92 |
| | RH closed with dessicant | | |

TABLE 7
HPLC 6 Week Closed Stability Data for
Spray Dried Dispersions of Compound 1

| | | Compound 1 | Impurities |
|---|---|---|---|
| | | Relative | Relative |
| | | abundance | abundance |
| | | (%) | (%) |
| Compound 1 | Stored at Room Temp. | 97.70 | 2.33 |
| 25/75 (w/w) | 6 weeks at 25° C./60% | 96.86 | 3.25 |
| Compound | RH closed with dessicant | | |
| 1/HPMCAS-M | 6 weeks at 40° C./75% | 96.34 | 3.80 |
| | RH closed with dessicant | | |
| 25/75 (w/w) | 6 weeks at 25° C./60% | 96.36 | 3.72 |
| Compound | RH closed with dessicant | | |
| 1/HPMCAS-L | 6 weeks at 40° C./75% | 95.89 | 4.21 |
| | RH closed with dessicant | | |
| 25/75 (w/w) | 6 weeks at 25° C./60% | 96.91 | 3.25 |
| Compound | RH closed with dessicant | | |
| 1/PVP-VA64 | 6 weeks at 40° C./75% | 96.87 | 3.21 |
| | RH closed with dessicant | | |
| 25/75 (w/w) | 6 weeks at 25° C./60% | 96.75 | 3.30 |
| Compound | RH closed with dessicant | | |
| 1/Eudragit ® | 6 weeks at 40° C./75% | 95.21 | 4.04 |
| L100 | RH closed with dessicant | | |
| 25/75 (w/w) | 6 weeks at 25° C./60% | 96.59 | 3.53 |
| Compound | RH closed with dessicant | | |
| 1/HPMC E3 | 6 weeks at 40° C./75% | 94.48 | 4.62 |
| | RH closed with dessicant | | |

### Example 6: Closed Stability Packaging Study of PVP-VA SDDs of Compound 1

Chemical stability of PVP-VA-containing SDDs of Compound 1 under closed conditions was evaluated by HPLC using the method provided herein in Table 4. Minimal degradation of the 25/75 (w/w) Compound 1 PVP-VA SDD was observed after three months of storage under closed condition. The six week analysis of the 25/70/5 (w/w/w) Compound 1/PVP-VA/Poloxamer 407 SDD also reveals good chemical stability. Stability data for PVP-VA containing SDDs of Compound 1 are provided in Table 8.

TABLE 8
Closed Stability Data For PVP-VA-Containing
Dried Dispersions of Compound 1

| | | Compound 1 | Impurities |
|---|---|---|---|
| | | Relative | Relative |
| | | abundance | abundance |
| | | (%) | (%) |
| Compound 1 | Stored at Room Temp. | 97.54 | 2.33 |
| 25/75 (w/w) | Stored at 5° C. | 97.14 | 2.86 |
| Compound | 3 months at 25° C./60% | 96.97 | 3.09 |
| 1/PVP-VA SDD | RH closed with dessicant | | |
| | 6 weeks at 40° C./75% | 96.92 | 3.11 |
| | RH closed with dessicant | | |
| 25/70/5 (w/w/w) | Stored at 5° C. | 97.37 | 2.63 |
| Compound 1/ | 6 weeks at 25° C./60% | 97.38 | 2.62 |
| PVP-VA/ | RH closed with dessicant | | |
| Poloxamer | 6 weeks at 40° C./75% | 97.29 | 2.74 |
| 407 SDD | RH closed with dessicant | | |

### Example 7: Tablet Formulations of Compound 1

Tablets were prepared by combining all intragranular materials (i.e., the active intermediate, ductile filler, brittle filler, disintegrant and glidant) except lubricant and blending the material in a 16 quart V-shell blender for 60 revolutions at a speed of 16 revolutions per minute (rpm) to form a pre-blend. The pre-blend was then delumped by blending through a Quadro Comil Model U5 using a 032R screen using a round mill impellor at a speed of 1500+/−100 rpm. The delumped material was then returned to the 16 quart V-shell blender and blended or 180 revolutions at 16 rpm to form a pre-granulation main blend. The pregranulation blends prepared according to this example had high bulk and tapped densities, and a relatively low Carr index, indicating that the pregranulation blends should have acceptable flow characteristics for roller compaction. The pregranulation blends were also found to be highly compressible, tabletable, and compactable.

The intragranular lubricant was added to the pre-granulation main blend, mixed manually for about 15-30 seconds and then passed through a #20 mesh screen back into the blender and mixed for 48 revolutions at 16 rpm to form the main lubricant pregranulation blend. The main lubricant pregranulation blend was roller compacted using a Gerties Mini-Pactor using the settings in Table 9 to form an intragranular blend.

TABLE 9
Roller Compaction Settings

| | |
|---|---|
| Parameters | Settings |

Roll Settings

| | |
|---|---|
| Master Roll (lower, left position) | Knurled |
| Second Roll (upper, right position) | Knurled |
| Compression Force (kN/cm) | 3.0 |
| Press Roller Start Speed (rpm) | 0.5 |
| Press Roller Speed (rpm) | 1.5 |
| Gap Width (mm) | 2.0 |
| PID (unitless:msec:msec) | 3:8000:0 |

Feed Settings

| | |
|---|---|
| Agitator Speed (rpm) | 5 |
| Feed Factor | 0.7 |
| Density Factor | 0.7 |
| Tamp/Feed Ratio | 180 |
| Gap-Control Activated (On/Off) | On |
| Torque Control (On/Off) | Off |

Granulator Settings

| | |
|---|---|
| Granulator Type | Pocket |
| Granulator Screen Type | Square-mesh |
| Granulator Screen Size (mm) | 1.00 |
| Granulator Rotor Block Setting (mm) | 1.0 |
| Granulator Speed (rpm) | 50 |
| Granulator CW/CCW Degrees^{a, b} | 240/360 |

^{a}CW = clockwise rotation; CCW = counter-clockwise rotation.

Extragranular disintegrant was added to the intragranular blend and blended in an 8 quart V-shell blender for 180 revolutions at a speed of 18 rpm to form an extragranular main blend. Extragranular lubricant was passed through a #20-mesh screen and added to the extragranular main blend and blended from 48 revolutions at 18 rpm to form an extragranular lubricant blend. The extragranular blends prepared according to this example had high bulk and tapped densities, and a relatively low Carr index, indicating that the pregranulation blends should have acceptable flow characteristics for roller compaction.

The extragranular lubricant blend was then compressed on a Korsch XL100 tablet press (9/32″ SRC tooling, Sotax ST50 tablet hardness tester) to form tablets with a target weight of 150 mg. The tablet press was adjusted to achieve target tablet weight and hardness as needed. The tablets were then dedusted with a vibratory and/or vacuum deduster. The compression parameters are provided below.

| | |
|---|---|
| Parameter | Setting |

Tablet Press Settings

| | |
|---|---|
| Tooling Size and Shape | 9/32″ SRC |
| Tooling drawing number | 91270 |
| Number of tooling stations | 5 |
| Turret Speed (rpm) | 20 |
| Fill Cam (mm) | 4-10 |
| Feed Frame Paddle Type | Standard |
| Dosing Depth | 5.90 |
| Pre-Compression Insertion Depth (mm) | Adjust as needed |
| Main Compression Insertion Depth (mm) | Adjust as needed |
| Main Compression Edge Thickness (mm) | 1.75 |
| Pre-Compression Force (kN) | 0.2 |
| Main Compression Force (kN) | 4.4 |
| De-duster | Yes - Post Compression |

Bulk Tablet Attribute Averages (n = 50)

| | |
|---|---|
| Weight (mg) | 150.4 |
| Tablet Thickness (mm) | 3.9 |
| Tablet Hardness (kp) | 7.62 |

Two rounds of formulation and manufacture were performed. The first round, “Round A”, focused on identification of formulations for use with a Compound 1/PVP-VA64 25/75 (w/w) spray dried dispersion. Brittle filler and disintegrant selections were varied in order to evaluate the formulations' disintegration times and stability performance. “Round B” of formulation assessed replacing lactose monohydrate with anhydrous lactose to assess if the different lactose forms resulted in different stability results within the tablets. Tablets A1, A2, A3, and B1 were determined to have acceptable friability and dissolution characteristics. The formulations are summarized in Table 10. Formulations C1 and C2 are further manufacturing batches and have the same composition as Formulation A1.

TABLE 10
Tablet Formulations of Compound 1

| Formulation | | | |
|---|---|---|---|
| A1/C1/C2 | A2 | A3 | B1 |
| Tablet weight (mg) | | | |
| 150 | 150 | 150 | 150 |
| Tablet Dose Target (mg) | | | |
| 5.0 | 5.0 | 5.0 | 5.0 |

| | | |
|---|---|---|
| Function | Ingredient | % of Blend |

Intragranular

| | | | | | |
|---|---|---|---|---|---|
| Active | 25/75 VK2809/PVP-VA64 | 13.30% | 13.30% | 13.30% | 13.30% |
| Intermediate | Spray Dried Dispersion | | | | |
| Ductile Filler | Microcrystalline Cellulose | 50.47% | 50.47% | 50.47% | 50.47% |
| | (Avicel PH102) | | | | |
| Brittle Filler | Lactose Monohydrate | 25.23% | 25.23% | — | — |
| | (Fastflo 316) | | | | |
| Brittle Filler | Anhydrous Lactose (NF) | — | — | — | 25.23% |
| Brittle Filler | Mannittol (mannogem EZ) | — | — | 25.23% | — |
| Disintegrant | Croscarmellose Sodium | 6.00% | — | 6.00% | 6.00% |
| | (Ac-Di-Sol SD-711) | | | | |
| Disintegrant | Crospovidone (PVP-XL) | — | 6.00% | — | — |
| Glidant | Colloidal silicon dioxide | 0.50% | 0.50% | 0.50% | 0.50% |
| | (Syloid 244 FP) | | | | |
| Lubricant | Magnesium Stearate | 0.25% | 0.25% | 0.25% | 0.25% |

Extragranular

| | | | | | |
|---|---|---|---|---|---|
| Disintegrant | Croscarmellose Sodium | 4.00% | — | 4.00% | 4.00% |
| | (Ac-Di-Sol SD-711) | | | | |
| Disintegrant | Crospovidone (PVP-XL) | — | 4.00% | — | — |
| Lubricant | Magnesium Stearate | 0.25% | 0.25% | 0.25% | 0.25% |

| | | | | |
|---|---|---|---|---|
| Totals | 100.00% | 100.00% | 100.00% | 100.00% |
| Percent polymer | 9.98% | 9.98% | 9.98% | 9.98% |

Tablets A1, A2, A3, and B1 were evaluated in a USP II sink dissolution test with 1 wt % sodium lauryl sulfate (SLS) at 50° C. All tablets show average release for formulations were adequate, with formulations A1, A2, and B1 having a release of greater than 80% after ten minutes.

### Example 8: Stability of Tablet Formulations

Tablet formulations were subjected to a stability study. Tablets were packaged in heat sealed HDPE bottles with minimal headspace and stored in temperature-controlled stability chambers at 50° C., 40° C., and 25° C., and 5° C. Humidity in the chambers was either controlled or allowed to be at ambient humidity. The conditions are listed for each particular formulation in Table 11 below.

An overall comparison of the tablet formulations suggests that formulation A1 is most chemically stable as it exhibited the least change in total impurities between t=0 and 1 month at either condition.

TABLE 11
Tablet Formulation Stability

| | | | | Compound 1 | Impurities |
|---|---|---|---|---|---|
| | | | | Relative | Relative |
| | | | | abundance | abundance |
| | Condition | Timepoint | | (%) | (%) |
| Compound 1 | Stored at Room Temp. | 2 month | | 97.22 | 2.78 |
| Standard | | analysis | | | |
| | | 3 month | | 97.45 | 2.55 |
| | | analysis | | | |
| 25/75 w/w | Stored at 5° C. | 2 month | | 97.23 | 2.77 |
| Compound 1/ | | analysis | | | |
| PVP-VA | 25° C./60% RH closed | 3 | months | 97.19 | 2.81 |
| Spray Dried | with dessicant | | | | |
| Dispersion | 40° C. closed, | 1 | month | 97.05 | 2.95 |
| | ambient RH | 3 | months | 96.64 | 3.36 |
| | 50° C. closed, | 1 | month | 97.05 | 2.95 |
| | ambient RH | 2 | months | 97.16 | 2.84 |
| | | 3 | months | 96.90 | 3.10 |
| Tablet | Stored at 5° C. | t = 0 | | 97.23 | 2.72 |
| Formulation A1 | 25° C./60% RH closed | 3 | months | 97.50 | 2.50 |
| | with dessicant | | | | |
| | 40° C. closed, | 1 | month | 97.23 | 2.77 |
| | ambient RH | 2 | months | 97.56 | 2.44 |
| | | 3 | months | 97.46 | 2.54 |
| | 50° C. closed, | 1 | month | 97.12 | 2.88 |
| | ambient RH | 2 | months | 97.29 | 2.71 |
| | | 3 | months | 97.25 | 2.75 |
| Tablet | Stored at 5° C. | t = 0 | | 97.26 | 2.66 |
| Formulation A2 | 25° C./60% RH closed | 3 | months | 97.47 | 2.53 |
| | with dessicant | | | | |
| | 40° C. closed, | 1 | month | 97.15 | 2.85 |
| | ambient RH | 2 | months | 97.45 | 2.55 |
| | | 3 | months | 97.32 | 2.68 |
| | 50° C. closed, | 1 | month | 97.02 | 2.98 |
| | ambient RH | 2 | months | 97.06 | 2.94 |
| | | 3 | months | 96.97 | 3.03 |
| Tablet | Stored at 5° C. | t = 0 | | 97.01 | 2.96 |
| Formulation A3 | 25° C./60% RH closed | 3 | months | 97.19 | 2.81 |
| | with dessicant | | | | |
| | 40° C. closed, | 1 | month | 96.85 | 3.15 |
| | ambient RH | 2 | months | 97.10 | 2.90 |
| | | 3 | months | 97.10 | 2.90 |
| | 50° C. closed, | 1 | month | 96.90 | 3.10 |
| | ambient RH | 2 | months | 96.98 | 3.02 |
| | | 3 | months | 96.97 | 3.03 |
| Tablet | Stored at 5° C. | t = 0 | | 96.99 | 2.99 |
| Formulation B1 | 25° C./60% RH closed | 3 | months | 97.10 | 2.90 |
| | with dessicant | | | | |
| | 40° C. closed, | 1 | month | 96.84 | 3.16 |
| | ambient RH | 2 | months | 97.01 | 2.99 |
| | | 3 | months | 96.95 | 3.05 |
| | 50° C. closed, | 1 | month | 96.64 | 3.36 |
| | ambient RH | 2 | months | 96.78 | 3.22 |
| | | 3 | months | 96.82 | 3.18 |
| Tablet | Stored at 5° C. | t = 0 | | 90.2 | 2.5 |
| Formulation C1 | | 3 | months | 95.1 | 3.1 |
| | | 6 | months | 94.9 | 3.0 |
| | | 9 | months | 94.9 | 3.2 |
| | | 12 | months | 92.3 | 3.0 |
| | 25° C./60% RH closed | t = 0 | | 95.1 | 3.1 |
| | with dessicant | 3 | months | 95.8 | 3.1 |
| | | 6 | months | 94.6 | 3.1 |
| | | 9 | months | 94. | 3.2 |
| | | 12 | months | 91.5 | 3.1 |
| | 40° C. closed/75% RH | t = 0 | | 95.1 | 3.1 |
| | closed with dessicant | 3 | months | 94.3 | 3.5 |
| | | 6 | months | 94.3 | 3.9 |
| | | 9 | months | 91.8 | 4.6 |
| | | 12 | months | 88.5 | 5.7 |
| Tablet | 25° C./60% RH closed | t = 0 | | 97.2 | 3.2 |
| Formulation C2 | with dessicant | 1 | month | 99.4 | 3.3 |
| | | 3 | months | 97.9 | 3.2 |
| | | 6 | months | 98.0 | 3.4 |
| | | 9 | months | 98.8 | 3.5 |
| | | 12 | months | 97.6 | 3.5 |
| | | 18 | months | 97.6 | 3.7 |
| | | 24 | months | 96.5 | 3.7 |
| | 40° C. closed/75% RH | t = 0 | | 97.2 | 3.2 |
| | closed with dessicant | 1 | month | 99.6 | 3.4 |
| | | 3 | months | 97.4 | 3.6 |
| | | 6 | months | 95.9 | 4.3 |
| Compound 1 | Stored at 5° C. | t = 0 | | 96.84 | 3.16 |
| (encapsulated PEG- | 5° C., ambient | 1 | months | 96.75 | 3.25 |
| based formulation)* | humidity | 3 | months | 96.40 | 3.60 |
| | | 6 | months | 96.36 | 3.64 |
| | | 9 | months | 96.73 | 3.27 |
| | | 12 | months | 95.96 | 4.04 |
| | 25° C./60% RH closed | 1 | months | 95.89 | 4.11 |
| | with dessicant | 3 | months | 93.77 | 6.23 |
| | | 6 | months | 91.28 | 8.72 |

*current clinical dosage form

With respect to the use of substantially any plural and/or singular terms herein, those having skill in the art can translate from the plural to the singular and/or from the singular to plural as is appropriate to the context and/or application. The various singular/plural permutations can be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An oral dosage form comprising:
(A) a composition comprising Compound 1 having the structure: 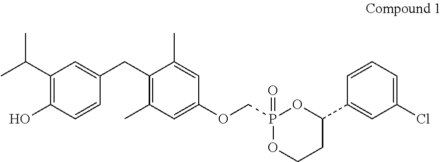
or a pharmaceutically acceptable salt thereof; and
one or more polymers selected from the group consisting of: polyvinyl pyrrolidone (PVP), polyvinyl pyrrolidinone-vinyl acetate copolymer (PVP-VA), polyvinyl alcohol (PVA), polyacrylic acid (PAA), poly(ethylene oxide) (PEO), hydroxypropyl cellulose (HPC), hydroxypropyl methyl cellulose (HPMC), copovidone, poloxamer 407, hypromellose acetate succinate (HPMCAS), polyacrylates and combinations thereof; and
(B) one or more ductile fillers, brittle fillers, disintegrants, glidants, lubricants, or combinations thereof.

2. The oral dosage form of claim 1, wherein the polymer is polyvinyl pyrrolidinone-vinyl acetate copolymer (PVP-VA).

3. The oral dosage form of claim 1, wherein Compound 1 and the one or more polymers are combined to form a spray dried dispersion.

4. The oral dosage form of claim 1, wherein Compound 1 and the one or more polymers are combined to form a hot melt extrusion.

5. (canceled)

6. The oral dosage form of claim 1, wherein the mass ratio of Compound 1 and the one or more polymers in the composition is from 1:1 to 1:4.

7. The oral dosage form of claim 6, wherein the mass ratio of Compound 1 and the one or more polymers in the composition is 1:3.

8. The oral dosage form of claim 1, wherein the composition comprises 5% to 25% by weight of the dosage form.

9. (canceled)

10. The oral dosage form of claim 1, wherein the ductile filler is selected from microcrystalline cellulose and silicified microcrystalline cellulose.

11. The oral dosage form of claim 1, wherein the ductile filler is microcrystalline cellulose.

12. The oral dosage form of claim 11, wherein the microcrystalline cellulose comprises 40% to 60% by weight of the dosage form.

13. (canceled)

14. The oral dosage form of claim 12, wherein the microcrystalline cellulose comprises 50% by weight of the dosage form.

15. The oral dosage form of claim 1, wherein the brittle filler comprises lactose monohydrate, spray dried lactose, anhydrous lactose, lactose monohydrate, anhydrous lactose, mannitol, and combinations thereof.

16. (canceled)

17. The oral dosage form of claim 1, wherein the brittle filler comprises 10% to 40% by weight of the dosage form.

18. (canceled)

19. The oral dosage form of claim 1, wherein the brittle filler comprises 25% by weight of the dosage form.

20. The oral dosage form of claim 1, wherein the disintegrant is selected from carmellose calcium, carboxymethylstarch sodium, croscarmellose sodium, starch, pregelatinized starch, sodium starch glycolate, crosslinked polyvinylpyrrolidone crospovidone, hydroxypropylcellulose, magnesium aluminometasilicate, and polacrilin potassium.

21. (canceled)

22. The oral dosage form of claim 1, wherein the disintegrant comprises 5% to 15% by weight of the dosage form.

23. The oral dosage form of claim 1, wherein the disintegrant comprises 10% by weight of the dosage form.

24. The oral dosage form of claim 1, wherein the lubricant is selected from the group consisting of stearic acid, talc, glyceryl behenate, sodium stearyl fumarate and magnesium stearate.

25. The oral dosage form of claim 1, wherein the lubricant is magnesium stearate.

26. (canceled)

27. The oral dosage form of claim 1, wherein the lubricant comprises 0.1% to 1% by weight of the dosage form.

28. (canceled)

29. The oral dosage form of claim 1, wherein the glidant is silicon dioxide, starch, or talc.

30. (canceled)

31. (canceled)

32. The oral dosage form of claim 1, wherein the glidant comprises 0.1% to 1% by weight of the dosage form.

33. (canceled)

34. The oral dosage form of claim 1 comprising:
(a) an intragranular portion comprising: (i) Compound 1, or a pharmaceutically acceptable salt thereof, and the one or more polymers; (ii) a ductile filler; (iii) a brittle filler; (iv) a disintegrant; (v) a glidant; and (vi) a lubricant; and
(b) an extragranular portion comprising a (i) disintegrant; and (ii) a lubricant.

35. The oral dosage form of claim 1, wherein the dosage form is characterized by having from 90% to 100% of the original amount of Compound 1 after 1 month of storage at 50° C. and ambient relative humidity (RH).

36. (canceled)

37. The oral dosage form of claim 1, wherein the dosage form is characterized by having from 90% to 100% of the original amount of Compound 1 after 2 months of storage at 50° C. and ambient relative humidity (RH).

38. (canceled)

39. A method of preparing an oral dosage of claim 1 comprising the steps of:
(a) preparing a pregranulation pre-blend comprising (a) a composition comprising Compound 1 and the one or more polymers; (ii) a ductile filler; (iii) a brittle filler; (iv) a disintegrant; and (v) a glidant;
(b) blending the pre-blend composition;
(c) further adding lubricant to the pregranulation pre-blend;
(d) slugging the pregranulation pre-blend; and
(e) granulating the pregranulation pre-blend to form granulated material.

40. (canceled)

41. A method of preventing, treating, or ameliorating one or more fatty liver diseases in a subject in need thereof comprising administering to said subject in need thereof, the method comprising administering the oral dosage forms of claim 1 to said subject.

42. (canceled)

43. (canceled)

44. (canceled)

45. (canceled)

46. (canceled)

47. (canceled)

48. (canceled)
